(19) United States
(12) Patent Application Publication
(10) Pub. No.: US 2015/0297082 A1
(43) Pub. Date: Oct. 22, 2015

(54) MEDICAL MONITORING SYSTEM

(71) Applicant: John M. HOGGLE, (US)

(72) Inventor: John M. HOGGLE, Douglasville, GA (US)

(21) Appl. No.: 14/647,279

(22) PCT Filed: Nov. 26, 2013
(86) PCT No.: PCT/US2013/071964

§ 371 Date: May 26, 2015

Related U.S. Application Data
(60) Provisional application No. 61/729,972, filed on Nov. 26, 2012.

(51) Int. Cl. A61B 5/00

(52) U.S. Cl. CPC A61B 5/0022; A61B 5/0026

(57) ABSTRACT

Various examples are provided for a medical monitoring. In one example, among others, a system for medical monitoring includes a monitoring device capable of obtaining one or more vital signs of a user and user equipment communicatively coupled to the monitoring device, the user equipment capable of obtaining the one or more vital signs from the monitoring device and providing the one or more vital signs to a remote server for access by a care giver. In another example, a system is configured to monitor the medical condition of a user and provide tasks to the user based at least in part upon the monitored medical condition. The tasks may be based at least in part based upon responses of the user to questions regarding the medical condition of the user.

### CROSS REFERENCE TO RELATED APPLICATIONS

This application claims priority to co-pending U.S. provisional application entitled “Medical Monitoring System” having Ser. No. 61/729,972, filed Nov. 26, 2012, the entirety of which is hereby incorporated by reference.

### BACKGROUND

Monitoring of a patient's vital signs to determine his or her condition is carried out in emergency room settings. Ambulances transport some of the monitoring capabilities and care givers to a patient, which improves the ability to respond quickly to the patient's condition. Upon arrival, a care giver can begin examining the patient to determine what, if any, treatment may be needed to address the patient's medical condition.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is an image illustrating an example of various components of a medical monitoring system in accordance with various embodiments of the present disclosure.

FIG. 2 is a diagram illustrating an example of the medical monitoring system 200 in accordance with various embodiments of the present application.

FIG. 3 is a diagram illustrating an example of the operation of the medical monitoring system of FIG. 2 in accordance with various embodiments of the present application.

FIGS. 4-16 are examples of various interface screens rendered on a display of user equipment of the medical monitoring system of FIG. 2 in accordance with various embodiments of the present application.

FIGS. 17-36 are examples of various interface screens rendered on a display of a client device of the medical monitoring system of FIG. 2 in accordance with various embodiments of the present application.

FIGS. 37A-37C are examples of a pain tracking screen rendered on a display of user equipment of the medical monitoring system of FIG. 2 in accordance with various embodiments of the present application.

FIG. 38 is an example of a pain care screen 606 rendered on a display of a client device of the medical monitoring system of FIG. 2 in accordance with various embodiments of the present application.

FIG. 39 is a schematic block diagram of an example of user equipment (UE) or client device 700 in accordance with various embodiments of the present disclosure.

FIG. 40 is a schematic block diagram of a computing device 800 according to an embodiment of the present disclosure.

### DETAILED DESCRIPTION

Disclosed herein are various examples related to medical monitoring. Reference will now be made in detail to the description of the embodiments as illustrated in the drawings, wherein like reference numbers indicate like parts throughout the several views.

Care of patients outside of a hospital or emergency room setting such as, e.g., at a battlefield or an accident scene provide challenges in medical monitoring and interventions, information capture, storage and security. Battlefield medics, first responders and other caregivers do not have access to physiological monitoring, medical information exchange, and imaging and telemedicine technologies. Without medical baseline and post injury monitoring, interventions are only as effective as the medic, first responder or other caregiver's medical knowledge allows. A mobile medical monitoring system can be used to provide physiological monitoring and telemetry, medical information exchange and analysis, diagnostic imaging and may be integrated into semi-autonomous, autonomous or closed-loop treatment systems. The mobile medical monitoring system may be used for care at the point of injury and/or during transport off the battlefield or from the scene of the accident. Continuous biomedical monitoring may be maintained throughout the medical evacuation process. The medical monitoring system may also be used for remote monitoring of patients suffering from congestive heart failure, pneumonia, acute myocardial infarxtion, or other acute and chronic illnesses after discharge from a hospital and/or while they are at home. Health systems involved in combat casualty care, internal development and disaster response, emergency medical services, wilderness medicine and/or health care in extreme environments may also utilize the system.

The mobile medical monitoring system utilizes wireless sensors and/or devices that monitor a person's vital signs. For instance, a harness including one or more wireless sensors and/or devices may be worn by or secured to a person to monitor vital information. The wireless sensors and devices communicate with user equipment (UE) such as, e.g., a smartphone, tablet or other appropriate mobile communication device. A client application (or App) running on the UE can support monitoring of the vital signs (or other patient information) by communicating with the sensor and devices using wireless communication technologies. For example, Bluetooth enabled devices can be communicatively coupled with an Android or Apple iOS based smartphone running the client application. The client application can provide a user interface to allow for local access to the person's monitored telemetry by the person and/or an on-site medic or care giver. In addition, the client application may allow the on-site medic or care giver to utilize information such as medical manuals or procedures (e.g., the Army Medical Handbook) and/or other care pathways for diagnosis and/or trauma treatment information. The client application may also communicate with web based application services to provide remote monitoring of the patient's vital telemetry. Communications may be carried out over civilian and/or military communications networks such as, e.g., tactical radio, broadband systems and cellular systems to provide a reliable carrier for the mobile medical monitoring system.

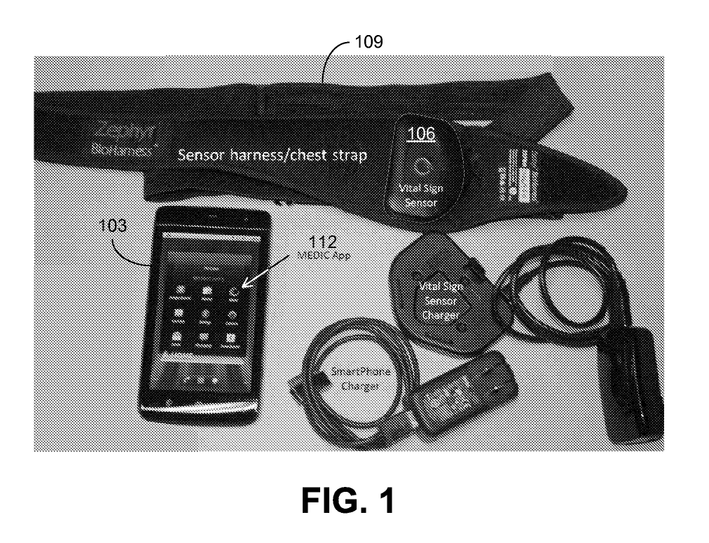

Referring to FIG. 1, shown is an example of various components of a medical monitoring system in accordance with various embodiments of the present disclosure. For example, the medical monitoring system includes user equipment (UE) 103 such as, e.g., a smartphone, tablet or other appropriate mobile communication device and a monitoring device 106 such as, e.g., a vital sign sensor that may be secured to a person (or patient) using, e.g., a sensor harness 109. In the example of FIG. 1, the sensor harness 109 is a chest strap that allows the monitoring device 106 to be secured in position about the chest of the person wearing the sensor harness 109. Other components such as, e.g., charging devices for the UE 103 and monitoring device 106 may be used to restore power levels of the UE 103 and monitoring device 106 as can be understood.

The UE 103 can support GPS (global positioning system), Bluetooth®, cellular, and/or WiFi (e.g., WLAN, wireless local area network) communications. The UE 103 can communicate with one or more monitoring devices 106 via, e.g., a Bluetooth® link. The monitoring device(s) 106 may be used to monitor conditions such as, e.g., heart rate, respiration rate, body orientation, activity level, body temperature, electrocardiograms (e.g., a single lead EKG or ECG), weight, blood pressure, glucose level, and/or oxygen level. One or more of the conditions may be monitored by a single monitoring device 106. A variety of wireless sensors may be utilized for remote medical diagnostics telemetry, including commercial off-the-shelf devices such as, e.g., the Zephyr Bio-Harness which provides heart rate, respiration rate, body orientation, body temperature and single lead EKG. Encrypted data may be securely sent from a monitoring device 106 to the UE 103 via a secure wireless link and/or personal area network (PAN) using Bluetooth®, ANT+, Ultra wide band (UWB) or other suitable wireless protocol via a secure wireless link and/or personal area network (PAN). A client application 112 executed on the UE 103 can facilitate local viewing, processing and/or forwarding of the data through a wireless connection with the UE 103. For instance, the UE 103 can establish a cellular and/or WiFi link to facilitate communication with a secure server and/or a care provider. The UE 103 can also provide a geophysical location of the person being monitored to facilitate patient monitoring and/or directions for rapid care and triage.

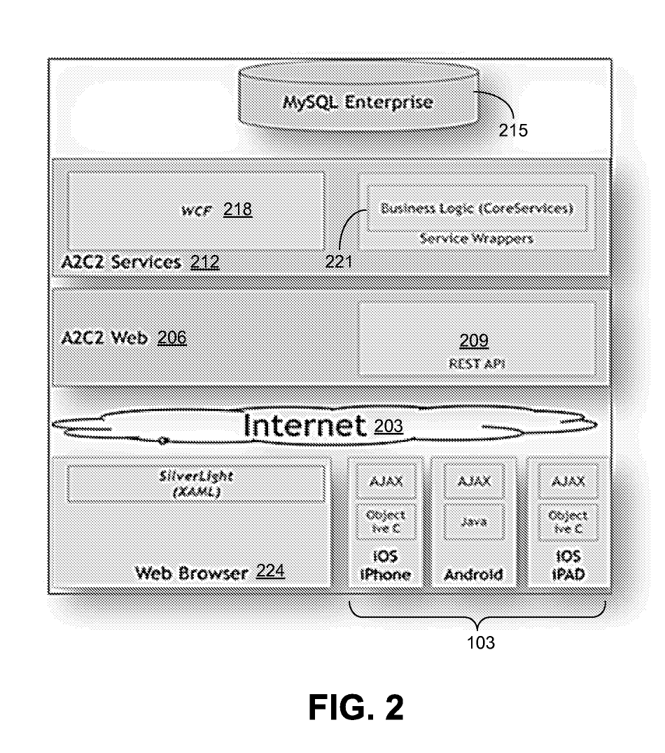

Referring now to FIG. 2, shown is a diagram illustrating an example of the medical monitoring system 200 in accordance with various embodiments of the present application. As discussed above, the medical monitoring system 200 includes one or more UEs 103 (e.g., communication devices utilizing Apple or Android operating systems) that communicate with one or more monitoring devices 106 configured to monitor conditions of the user of the UE 103. The monitoring devices 106 monitor the person's vital signs and communicated the data to the UE 103 through a wireless link. For example, Bluetooth® enabled devices are coupled with an Android or Apple iOS based smartphone running a client application, which may be implemented using Java, AJAX, C or other appropriate script or programing language. The UEs 103 can communicate data through, e.g., JSON (Javascript object notation) to collection REST (representational state transfer) web based application services to allow care providers to remotely monitor a patient's vital sign telemetry. A UE 103 can establish a wireless link through a communication network (e.g., a cellular network or WLAN) to communicate the data to a secure server and/or a care provider through, e.g., the internet 203.

The medical monitoring system 200 may be an SOA (service-oriented architecture) built to provide Enterprise SaaS (software as a service) care plan management. With the medical monitoring system 200 as the base care management system it is possible to develop a remote tele-medicine and remote medical monitoring program within a care network. Applications that fulfill the needs of the different agencies and departments and their customers or users of the services can be easily integrated with the medical monitoring system 200 in order to expand services.

A web based application 206 such as, e.g., a .Net application running in a virtual runtime environment can facilitate communication of the data between the UE 103, a secure database, and/or the care provider. The web based application 206 can include a REST API (application programming interface) 209 and/or web applications such as, e.g., a Silverlight client application to allow for cross platform and browser compatibility. A web application server can provide a .Net and C# service execution environment for the web based application 206. For example, Silverlight can provide a web portal solution for information access, application configuration and reporting and a REST application can provide a web service interface for mobile client applications.

An intermediate services layer 212 can operate between the web based application 206 and a secure database 215 for storing the communicated data. The services layer 212 may be .Net services running on, e.g., a Windows server, which can support data access and other services. For example, the services may utilize Windows Communication Foundation (WCF) 218 or other business logic 221. The services layer 212 can provide a backend C# services platform for services (server side) using WCF based Service Wrappers (.Net Classes) for core business logic services such as, e.g., tracker alert processing, GPS processing, notifications and vitals diagnostic processing.

The database 215 may be a MySQL Enterprise server running, e.g., on Linux or Windows server. The database 115 can drive functions of the medical monitoring system 200 from handling medical telematics data and business logic thresholds to patient and account permissions and service access credentialing. The database 115 may be configured for high availability and distributed data collection, synchronization and archiving. Existing databases such as medical manuals or procedures (e.g., the Army Medical Handbook) and/or other diagnostic or trauma treatment information may also be integrated with the medical monitoring system 200. In some implementations, predictive models may be used to identify the proper course of procedure for a given medical situation, which may be based upon the database information.

Care providers and/or managers may access the medical monitoring system 200 through a web browser 224 running on a client device such as, e.g., a tablet, smartphone, computer, etc. The web based application 206 provides a central hub and management interface for the medical monitoring system 200. A portal may be provided by a Silverlight application, which may be installed as a plugin for popular internet browsers (e.g., Microsoft Internet Explorer, Mozilla Firefox, Google Chrome, Apple Safari, etc.) The portal can provide care managers (or clinicians) the ability to create network organizations, vital trackers, care plans, schedules, as well as care plan tasks such as scheduling medications, visits, and education. Assessment tools along with questionnaires and specific tasks can be communicated to the patient UE 103 for completion.

The portal may also provide geo-spatial awareness via an interface display such as, e.g., an integrated Google Earth™ view. This can provide field medics and first responders a tactical view of a monitored person's location in order to quickly locate and track the patient. The portal can also utilize the WCF services to make accessible business logic of the medical monitoring system 200. The application logic includes a set of classes on top of the .NET framework's common language runtime (CLR) that may be made accessible to other software running both inside and outside the medical monitoring system 200 via SOAP (simple object access protocol), via a WCF-specific binary protocol, Windows message queuing, JSON REST web services and/or other network and application protocols.

In some implementations, the architecture of the web based application 206 can be access agnostic to support any IP broadband wired or wireless access network. Client applications can utilize the UE 103 (e.g., Android or iOS enabled smartphone or tablet) to access wide area network (WAN) wireless broadband connections or local area network (LAN) wireless connections for Internet service provider (ISP) access. Examples of suitable access networks used include, but are not limited to, the Oceus 3G QuicLINK, 4G LTE Xiphos, or any commercial cellular (e.g., 3G/4G) or wired broadband access network or home WiFi router. The medical monitoring system 200 may also utilize a tactical military radio environment, which may function in a less robust manner due to bandwidth constraints of shared use tactical radio capacity.

The client application 112 running on the UE 103 may be, e.g., a client Java application (e.g., Android Java) that provides a communication hub for an individual user's PAN for the monitoring devices 106. The client application 112 provides the internet gateway access point to servers of the medical monitoring system 200 for vitals telemetry processing, diagnosis and distribution. A client device used by, e.g., a medic or first responder can include a client application such as, e.g., a client Java application (e.g., Android Java) that provides field medical personnel and/or first responder(s) access to the client vitals telemetry monitored by the medical monitoring system 200. Users can locally access on their client device (e.g., smartphone or tablet) the information, administer assessments and communicate actions or record treatments and assessments. For a tablet, the interface is optimized for to take advantage of the larger screen dimensions.

Secure information transfer can be provided in a variety of ways. Secure transmission can be provided by a security application such as, e.g., CoreGuard's WindTalker™, which can support communications network information assurance and security needs. Data can be completely encrypted through CoreGuard's DISA (Defense Information System Agency) approved WindTalker™ security platform. CoreGuard provides cyber security solutions that enable commercial organizations to protect sensitive information, including but not limited to: privacy information, financial data, trade secrets, and intellectual property. The medical monitoring system 200 may be a DISA (Defense Information System Agency) security technical implementation guide (STIG) and joint interoperability test command (JITC) certified security application as a component of the JOLTED (joint operational long term evolution deployable) TACTICS (tactical cellular system) JCTD (joint capability technology demonstration) sponsored by the Joint Staff J6 and SOCOM (Special Operations Command). The medical monitoring system 200 may also be HIPPA (Health Insurance Portability and Accountability Act) approved.

While other forms of security applications may be used, the WindTalker™ security platform provides evolutionary cross-domain information security at the sub-document data-level, independent of hardware. It maintains native file formats, and protects data at all times, from creation to deletion including data at rest or in storage (including temporary files), in transmission, in use, across multiple networks and domains, across the Internet or in distributed systems such as, e.g., the “Cloud.” CoreGuard's cutting edge WindTalker™ security platform is the first cybersecurity system to embed information security protection at the data and sub-document level. WindTalker™ was designed for use by the U.S. Military and provides a high level of information security while enabling secure differential sharing and automated redaction.

The medical monitoring system 200 may be integrated into existing military and/or governmental communication networks including mobile network infrastructures that are used to deploy wireless communication solutions. The medical monitoring system 200 may also be utilized for remote monitoring of acute and chronic patients at home, facilitating active management of post discharge patient care which can reduce readmission rates. Individual patient care plans and/or vital patient information may be customized to manage the patient's care. The medical monitoring system 200 can allow for automated or real-time communication via the UE 103 to facilitate medication compliance, assessments, education, appointments and/or questionnaires. Real-time communication includes delays associated with the transmission of data from the monitoring device 106 to the user, medic, first responder, care giver or care provider Alerts may be generated for remote care providers (e.g., nurses and/or clinicians) when a value for a vital sign falls at and/or outside defined limits or thresholds of the patient care plan. The medical monitoring system 200 may also allow for real-time bidirectional audio and/or video communication between a user of the UE 103, medic, first responder, care giver and/or care provider. The geophysical location of the patient can assist medics and first responders to quickly locate the patient in situations where emergency services are needed. In many cases, the care of a plurality of patients may be managed in a concurrent fashion using the medical monitoring system 200.

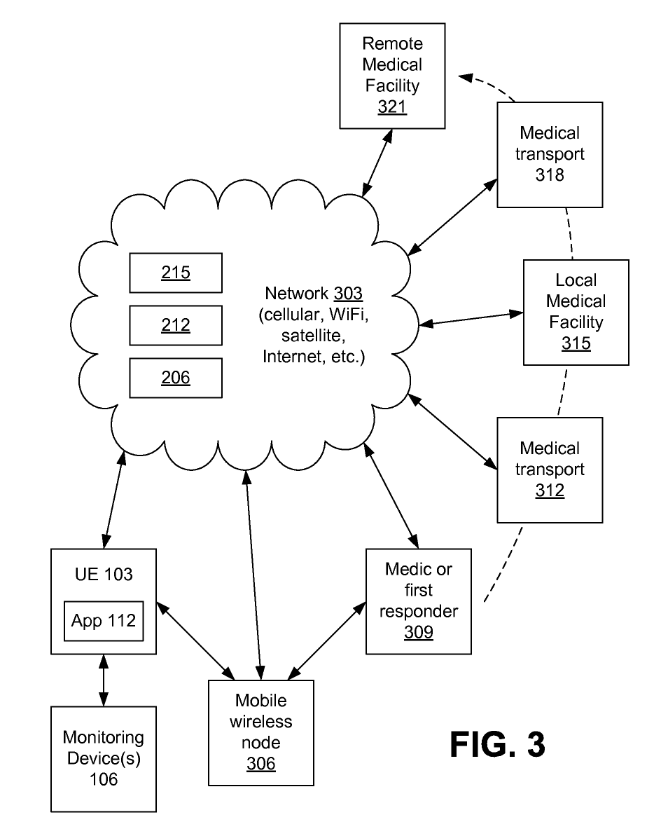

Referring next to FIG. 3, shown is an example of the operation of the medical monitoring system 200. To begin, a monitored user initiates operation of the client application 112 on the UE 103 by, e.g., selecting an icon displayed by the UE 103. The App 112 causes the UE 103 to pair with one or more monitoring device(s) 106 associated with the user via a wireless link (e.g., Bluetooth® or ANT+) or a PAN. The monitoring device(s) 106 can be turned on before starting the App 112. For example, the monitoring device 106 may have been charged, installed in a sensor harness 109 (FIG. 1), turned on and positioned on the user. Vital signs of the user may then be reported to the UE 103 by the monitoring device(s) 106 via the wireless link. An indication that the wireless link has been established may be indicated by the App 112. For instance, the current status of the monitored vital signs may be displayed by the UE 103. If a wireless link is not established (e.g., after a predefined time period), an indication may be provided to prompt the user to check the monitoring device 106. When multiple monitoring devices 106 are within range of the UE 103, the appropriate monitoring device(s) 106 may be identified by the UE 103 by verifying an identifier (ID) associated with the monitoring device 106.

The App 112 also causes the UE 103 to establish a cellular connection with network 303. In some cases, a WiFi connection may be established with the network 303 in place of the cellular connection. The network 303 can include access networks such as, e.g., Oceus 3G QuicLINK, 4G LTE Xiphos, or other cellular (e.g., 3G/4G or backhaul) or broadband (e.g., WiFi/WLAN) access networks, as well as the Internet. The user may login through the monitoring system portal to establish communications with the web based application 206 (FIG. 2). The UE 103 may then begin sending vital sign information for storage in the database and/or communication to medics, first responders, and/or care givers. In some cases, the cellular or WiFi connection may be established with the network 303 via a mobile wireless node 306 such as, e.g., a tactical cellular node.

A medic or first responder 309 may also establish a cellular (or WiFi) connection with then network 303 to access medical information associated with the user of the UE 103. The medic or first responder 309 may login with a client device such as, e.g., a tablet, smartphone, or laptop through the monitoring system portal to access the information. When a mobile wireless node 306 is used, the medic or first responder 309 may establish a connection via the mobile wireless node 306. For example, a combat medic may be able to monitor the condition of a squad of soldiers through the client device. In some cases, the vital information sent from the UE 103 may be sent directly to the client device of the medic or first responder 309 via the mobile wireless node 306 to reduce communication delays. If an emergency situation exists, the medic or first responder 309 can find the user of the UE 103 based upon the geophysical location provided by the UE 103 and begin triage and/or treatment using the vital sign telemetry sent to the client device of the medic or first responder 309.

In addition to the vital telemetry, information that may aid in the evaluation and/or treatment of the user of the UE 103 may also be sent to and/or accessed by the medic or first responder 309 via the client device. In some cases, remote care providers may assess the medical information of the user and send recommendations and/or instructions to the medic or first responder 309 regarding, e.g., treatment of the injury. The medic or first responder 309 may also be able to transmit medical information to the database 215 and/or to other care givers using the client device. In some cases, the client device may be configured to convert information spoken by the medic or first responder 309 to data (voice to text conversion), which may then be transmitted for storage and/or access from the database 215.

Medical transports and/or facilities may also access medical and location information of the user of the UE 103. For example, medical transport 312 (e.g., ambulance, helicopter or other evacuation vehicle) in route to the user may access the geophysical location provided by the UE 103 to determine the route to the injured user. In addition, the medical information provided by the UE 103 and/or the medic or first responder 309 may be accessed by the medical transport 312 to prepare for receiving the injured user. In this way, evacuation time and treatment may be optimized when every minute counts.

A local medical facility 315 such as, e.g., a combat or emergency response field hospital may also access medical and location information of the user of the UE 103. The information may improve triage of incoming patients. Access to the medical information of the injured user also allows care providers (e.g., nurses or clinicians) at the local medical facility 315 to provide feedback regarding treatment of the user to the medic or first responder 309 or personnel in the medical transport 312. Medical information may also be provided by care providers at the local medical facility 312 as the injured user is treated at the local medical facility.

Similarly, medical transport 318 from the local medical facility 315 to a remote medical facility 321, as well as the remote medical facility 321, may access the medical information of the user in a similar fashion as medical transport 312 and local medical facility 315. In this way, the user of the UE 103 may be monitored throughout transport from the original location to, e.g., the remote medical facility 321. Access by a remote medical facility may allow a specialist to consider the medical information and provide guidance regarding treatment of the injured user.

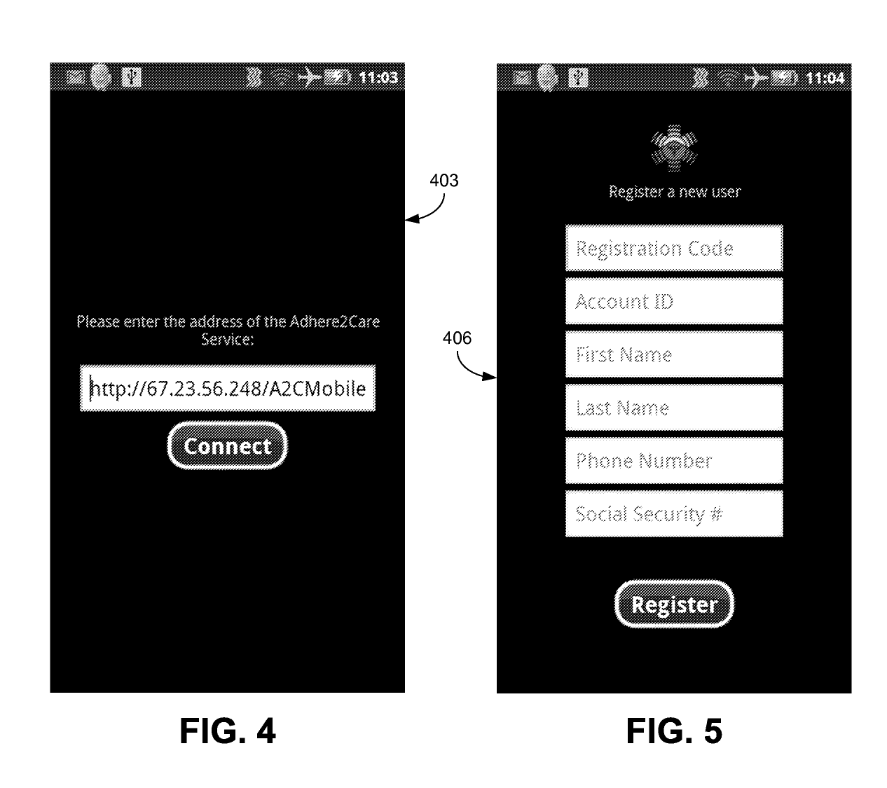

Referring now to FIGS. 4-16, shown are examples of client application screens rendered on a display of the UE 103 (FIGS. 1-3). Beginning with FIG. 4, a server connect screen 403 allows a user of the UE 103 to initiate connection with the web based application 206 (FIGS. 2-3) by entering the web address corresponding to the medical monitoring system 200 (FIG. 2). By entering the web address and selecting the “connect” icon, a link may be established to allow for communications with the web based application 206, intermediate services layer 212 and/or database 215 (FIGS. 2-3). The link may be established as a secure link. A new user may register an account with the medical monitoring system 200 through a registration screen 406 such as the example depicted in FIG. 5. A registration code and account ID, as well as other identifying information such as, e.g., first and last name, telephone number and social security number may be requested for proper classification of the new user. Other information may also be requested as part of the registration process. One or more additional screens (not shown) may be used to obtain the registration information. The user may initiate the registration process by selecting, e.g., a “register” icon on the UE display.

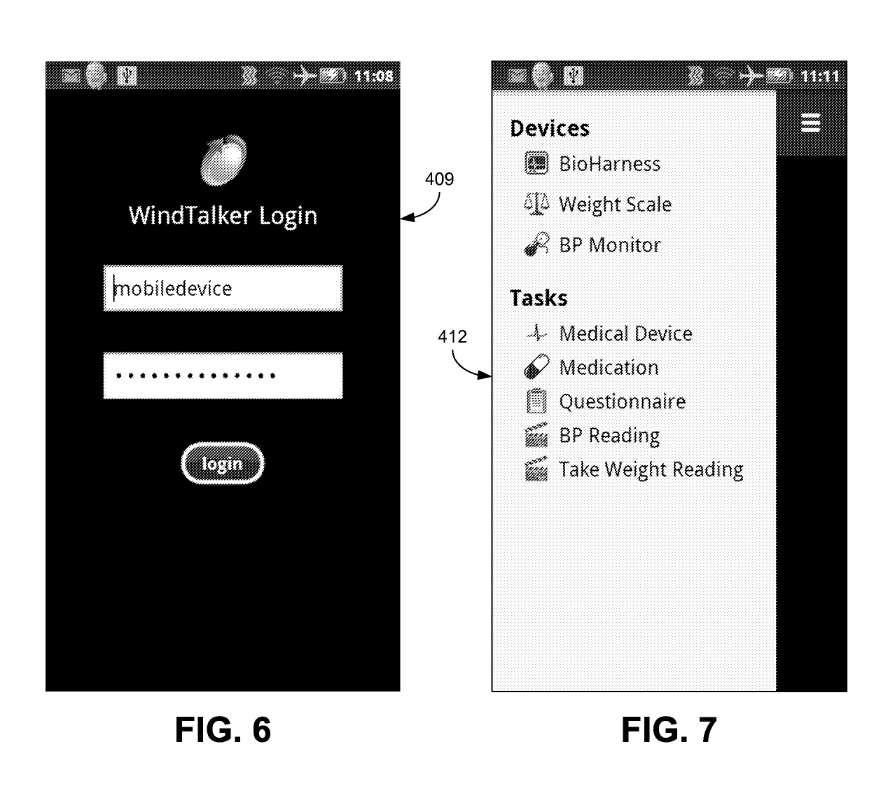

Once a connection has been established, an existing user of the medical monitoring system 200 may establish a secure encrypted link using a login screen 409 such as the example of FIG. 6. For example, CoreGuard's WindTalker™ may be used to securely communicate a user name and password to gain access to the medical monitoring system 200. The login process may be initiated by selecting the “login” icon after entering the appropriate information. After logging in, the client application 112 (FIGS. 1 and 3) provides a main menu screen 412 on the UE 103 such as the example depicted in FIG. 7. The main menu screen 412 may be customized for the user of the UE 103.

As illustrated in FIG. 7, the main menu screen 412 includes a devices section listing medical monitoring devices 106 (FIGS. 1-3) in communication with the UE 103 through a wireless connection such as, e.g., Bluetooth® or ANT+. In FIG. 7, the medical monitoring devices 106 include a monitoring device 106 secured on the user by a harness (or “bioharness”), a scale for obtaining weight of the user, and a blood pressure (BP) monitor. The main menu screen can also include a tasks section indicating tasks to be performed by the user of the UE 103. The tasks can include, e.g., adding a medical device 106 for monitoring of the user, taking medication, completing a questionnaire, taking readings of the user's condition with an existing monitoring device 106, or other tasks as can be understood. Tasks (or instructions) may be sent to the user by a care provider (e.g., nurse or clinician) through the portal of the web based application 206. The tasks may be prioritized by the care provider, user and/or based upon predefined criteria. By selecting the appropriate icon, the user may indicate completion of the task through a screen of the client application 112. The main menu may include an indication (e.g., a check mark after the task) that the task has been completed.

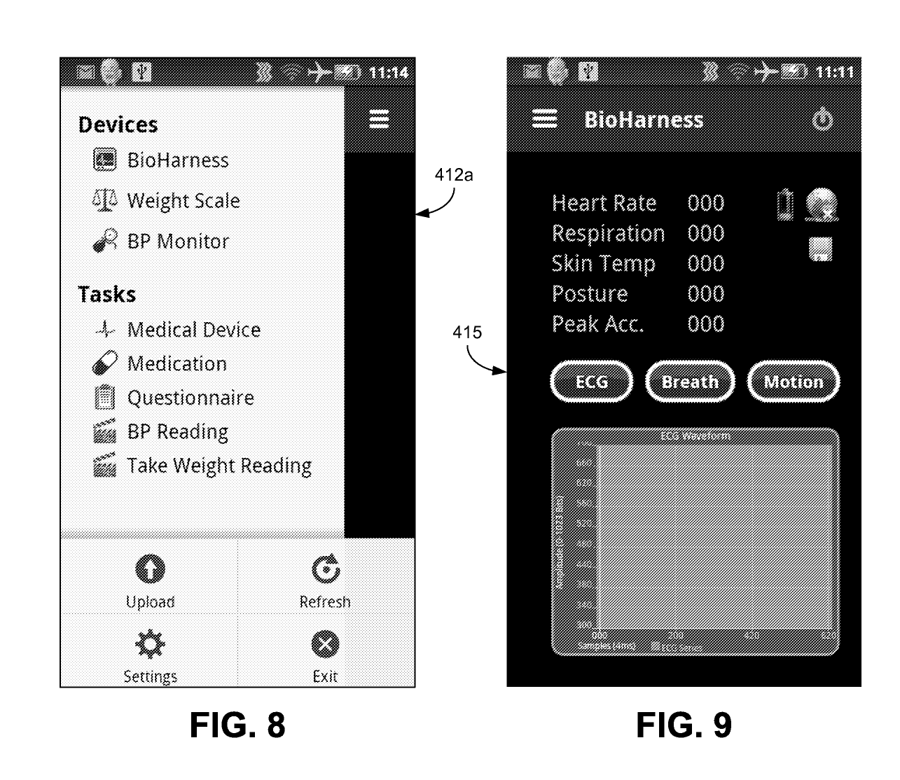

Other options may be available through the main menu screen 412. FIG. 8 shows an example of a main menu screen 412a with options. The main menu screen 412a can allow for uploading information to, e.g., the database 215 (FIG. 2) located on a remote server in the network 303 (FIG. 3). The main menu screen 412a may also allow for refreshing the status of the devices and tasks, e.g., by selecting a “refresh” icon. Settings for the client application 112 running on the UE 103 may be set or modified through the main menu screen 412a. The main menu screen 412a facilities exiting the client application 112 through an “exit” icon.

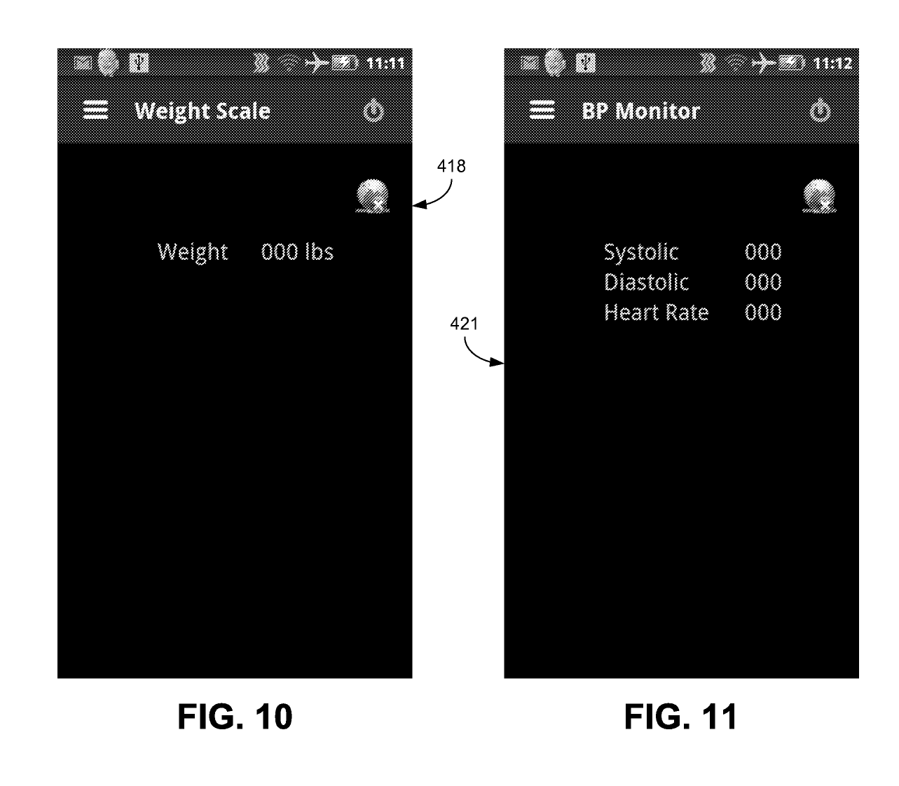

Data received from a monitoring device 106 may be accessed through a sensor screen by selecting the name of the device from the list in the devices section of the main menu screen 412. For instance, selecting the harness mounted sensor (or “bioharness”) causes the corresponding sensor screen 415 to be displayed on the UE 103. FIG. 9 shows an example of the sensor screen 415 for the monitoring device 106 secured to the user by a harness. The sensor screen 415 can display information from the monitoring device 106 in real-time. For example, heart rate (beats/min), respiration (breaths/min), skin temperature (° F. or ° C.), posture (zero degrees when standing upright), and/or peak acceleration associated with movement of the user may be indicated. Real-time waveforms for ECG, breathing and/or movement (in the two dimensional (2D) plane of the monitoring device 106) may be indicated on the screen by selecting the appropriate icon. The real-time waveforms may also be sent by the UE 103 for storage in the database 215 and/or access by a remote care provider. Other indications may also be provided such as, e.g., battery life of the monitoring device 106.

FIG. 10 shows an example of a sensor screen 418 for a weight scale. The sensor screen 418 can allow the user to obtain a weight measurement from a scale in communication with the UE 103 through, e.g., a Bluetooth® or other wireless link. FIG. 11 shows an example of a sensor screen 421 for blood pressure monitoring. The sensor screen 421 can allow the user to view and/or obtain blood pressure and/or pulse information of the user from a blood pressure monitor in communication with the UE 103 through, e.g., a Bluetooth® or other wireless link. Other interfacing options may be available through the sensor screens 418 and/or 421.

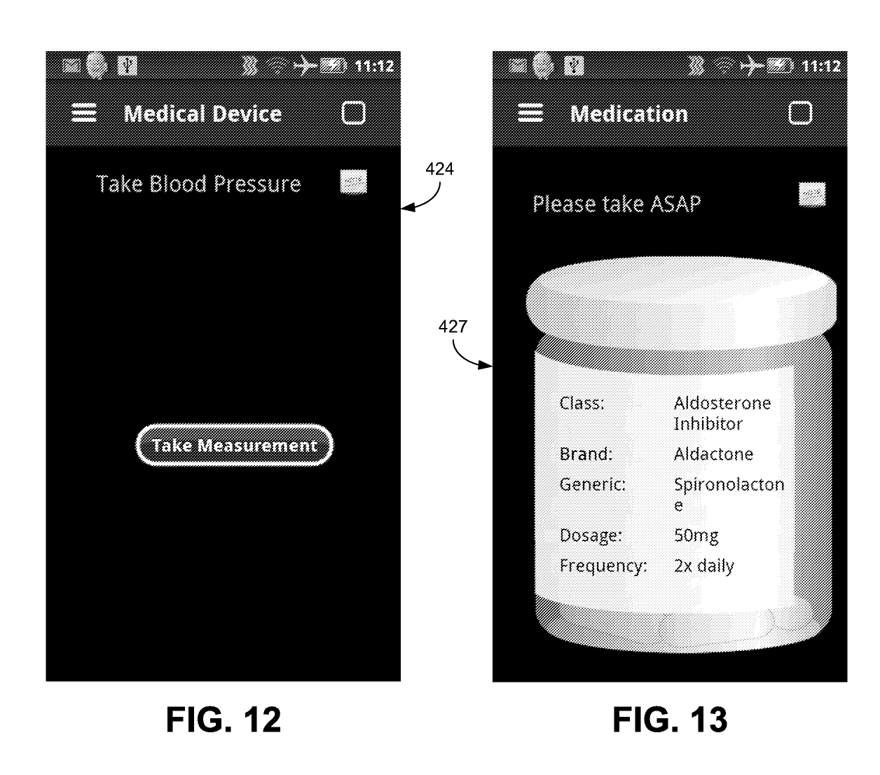

Task screens may also be accessed through the main menu screen 412 by selecting the name of the task from the list in the tasks section. For example, FIG. 12 shows a task screen 424 for taking blood pressure using the appropriate monitoring device 106. Selection of the “take measurement” icon can initiate acquisition of the information from the monitoring device. The task screen 427 shown in FIG. 13 is an example of a medication reminder. The task screen 427 prompts the user to take scheduled medication. Information provided in a task screen 427 may include the ype of medication and the dosage. One or more reminder may be scheduled by the user or for the user by a care provider. Notes may also be added to the task screens 424 and 427 by and/or for a care provider (e.g., nurse or clinician).

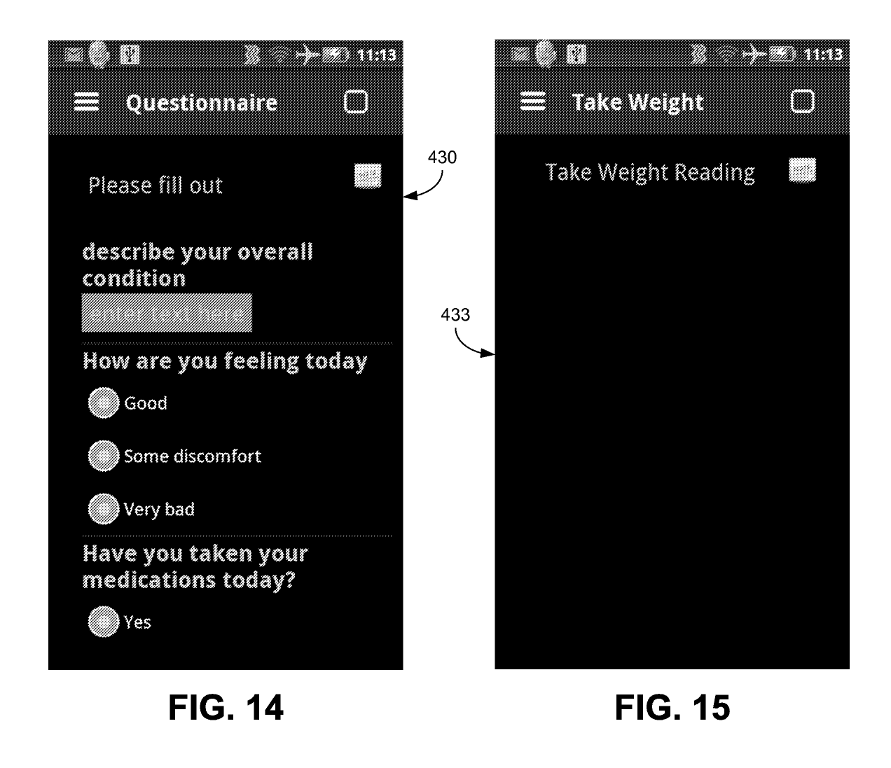

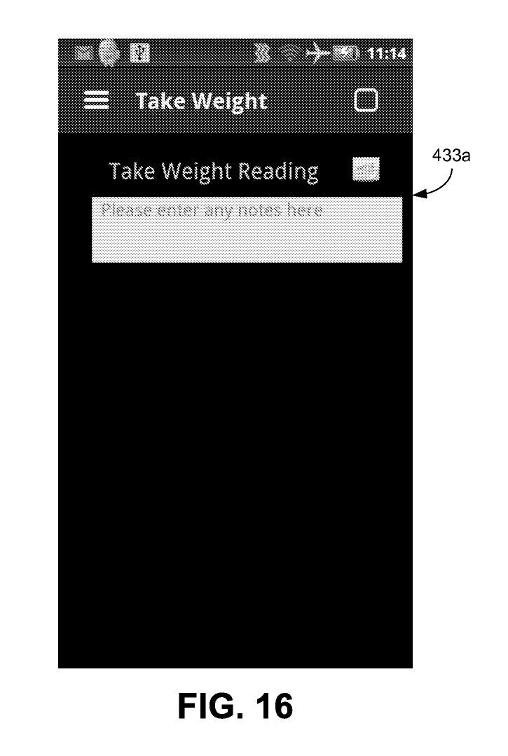

The task screen 430 in FIG. 14 is an example of a questionnaire to be completed by the user of the UE 103. The task screen 430 prompts the user for answers to care provider (e.g., nurse or clinician) questions. A plurality of questions may be provided to the user to aid in determining the condition of the user. As the care provider acquires more information, other questions may be generated by the care provider to further clarify the user's condition. Questions may take the form of yes/no questions, multiple choice questons, and/or short answer questions. FIG. 15 shows an example of a task screen 433 directing the user to take a weight reading. The task screen 433 allows the user to complete the requested task. Notes may also be added to the task screens 424 and 427 by and/or for a care provider (e.g., nurse or clinician). The user can add a note to an input area by selecting a “note” icon as illustrated by the task screen 433a of FIG. 16.

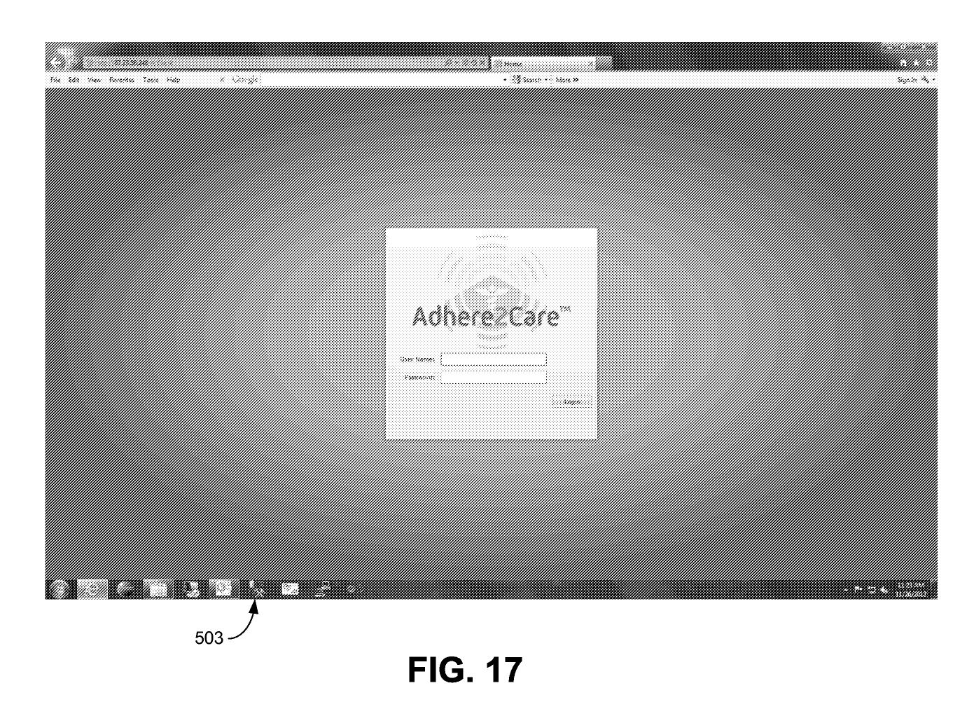

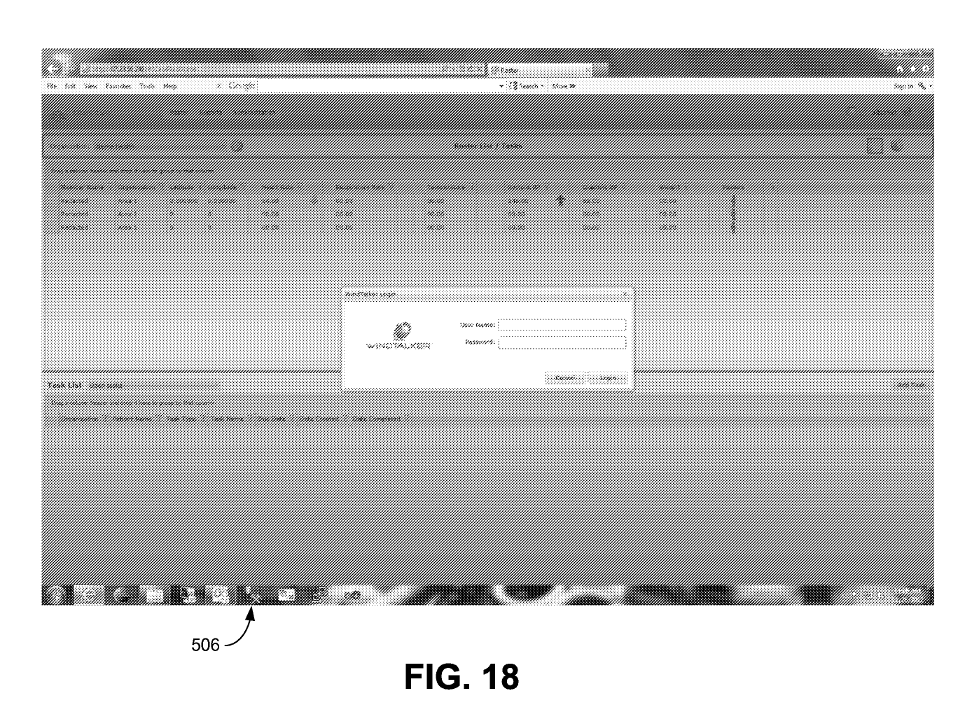

Referring now to FIGS. 17-36, shown are examples of web browser screens rendered on a display of a client device used by a medic, first responder, care giver or care provider. FIG. 17 shows an example of a login screen 503 that can be used to access patient information through the medical monitoring system 200. A medic, first responder, care giver or care provider can initiate a connection with the web based application 206 (FIGS. 2-3) by entering the web address corresponding to the medical monitoring system 200 (FIG. 2) through a web browser 224 (FIG. 2). The link may be established as a secure link. The medic, first responder, care giver or care provider may the link by entering a user name and password through the login screen 503 of FIG. 17. Secure access to information in the medical monitoring system 200 may be provided by, e.g., CoreGuard's WindTalker™ through a login screen 506 such as the example of FIG. 18. WindTalker™ allows for secure access of user medical information by the medic, first responder, care giver or care provider.

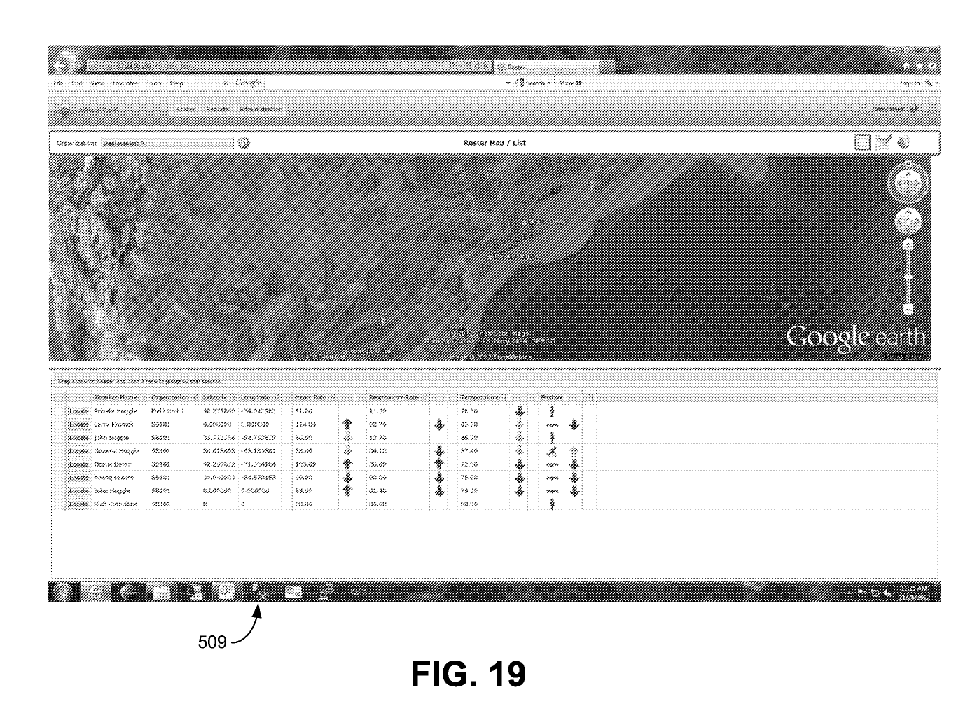

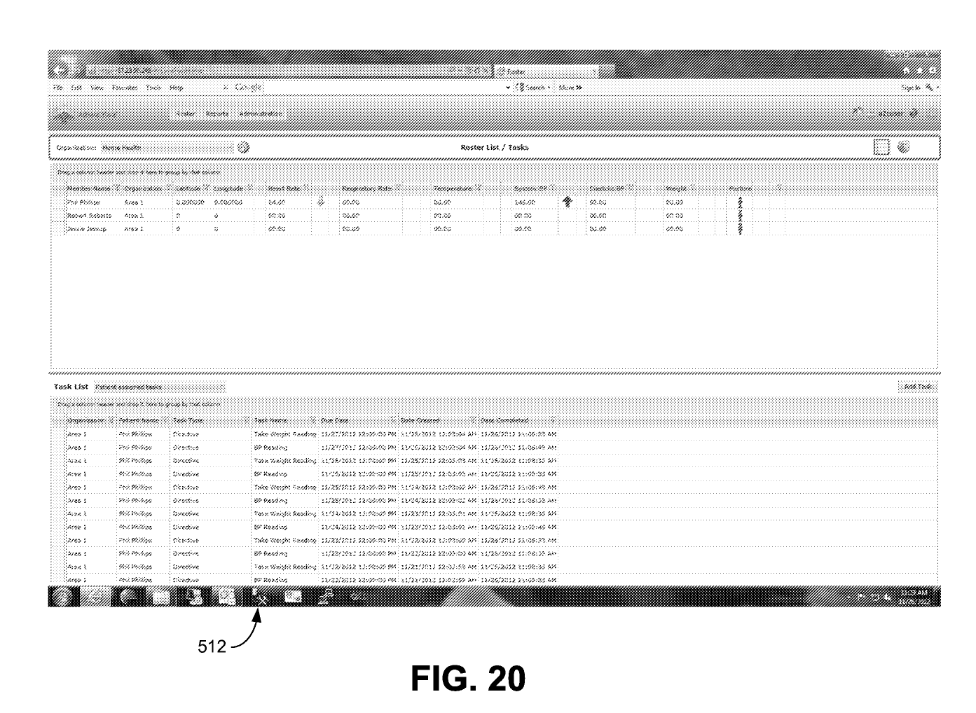

The medic, first responder, care giver or care provider can monitor the condition of a plurality of users through a web browser screen 509 such as the example of FIG. 19. For example, the geophysical location of the persons being monitored may be indicated through a map display such as, e.g., Google Earth. The scale of the map display may be adjusted to identify the location of a monitored user. The web browser screen 509 of FIG. 19 also includes a roster listing indicating various monitored conditions of the user. The roster listing includes, e.g., identifying information (e.g., name and organization), location information (e.g., latitude and longitude), and vital signs (e.g., heart rate, respiratory rate, temperature, and posture) for each of the monitored users. Graphical indications of changes in the vital signs can be provided by arrows, which may be color coded to indicate the extent of the change or a relationship to a defined threshold. FIG. 20 shows an example of a web browser screen 512 with a roster listing including a task list showing the task history of the selected user being monitored. The task history can include, e.g., the patient name, type of task (e.g., directive, questionnaire, etc.), task name, and time history (e.g., due date/time, creation date/time, and/or completion date/time).

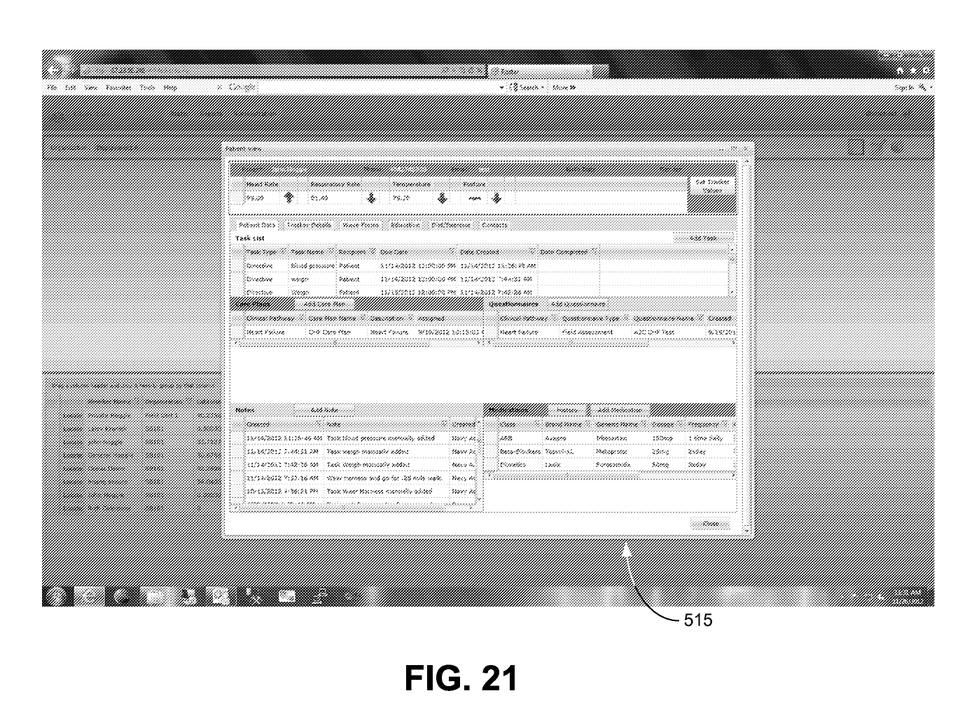

A patient view screen may be accessed by selecting one of the monitored users from the roster listing of FIG. 19. FIG. 21 shows an example of a patient view screen 515 accessible to the medic, first responder, care giver or care provider. The patient view screen 515 lists patient data such as, e.g., tasks, care plan, questionnaires, notes, and/or medication history associated with the selected user. Medical records can be accessed by the medic, first responder, care giver or care provider for evaluation of the user and/or modification of the care plan and/or medications. Patient vital signs being tracked may also be modified by the medic, first responder, care giver or care provider through the patient view screen. Other data such as, e.g., tracker details, captured waveforms, education, diet and/or exercise and/or contacts may also be accessed through the patient view screen 515. Tasks, medications, care plans, questionnaires, and/or notes may be added or modified through the patient view screen 515 by selecting the appropriate icon.

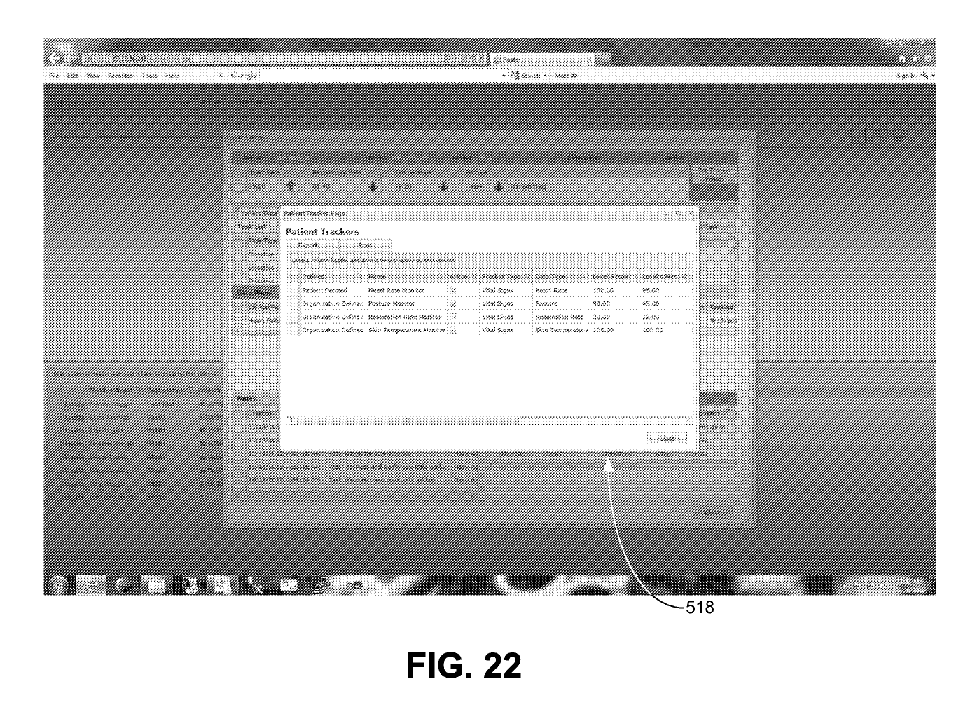

By selecting the appropriate icon in the patient view screen 515 (e.g., a “set tracker values” icon), the medic, first responder, care giver or care provider can access a patient tracker screen to review and/or modify the vital signs being tracked for that user (or patient) being monitored. FIG. 22 shows an example of a patient tracker screen 518. The patient tracker screen 518 includes a list of vital signs being tracked such as, e.g., heart rate, respirations rate, skin temperature, and/or posture of the selected user. The patient tracker screen 518 indicates, e.g., the source of the tracking request, the vital sign being tracked and the source of the data, whether the tracking is active or inactive, and threshold values (e.g., defined maximum and/or minimum limits) associated with each vital sign.

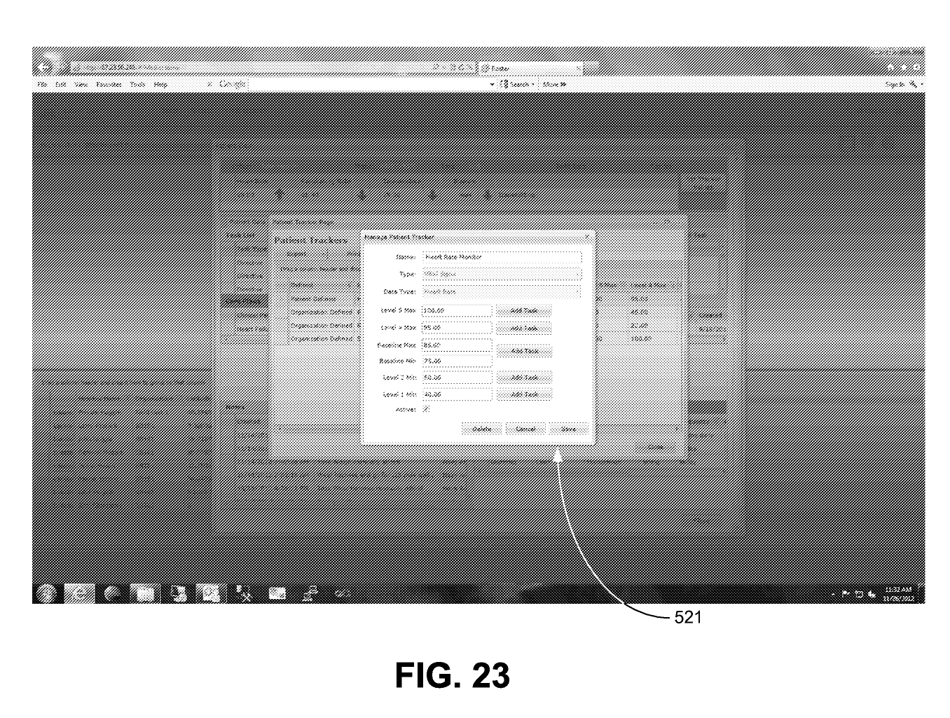

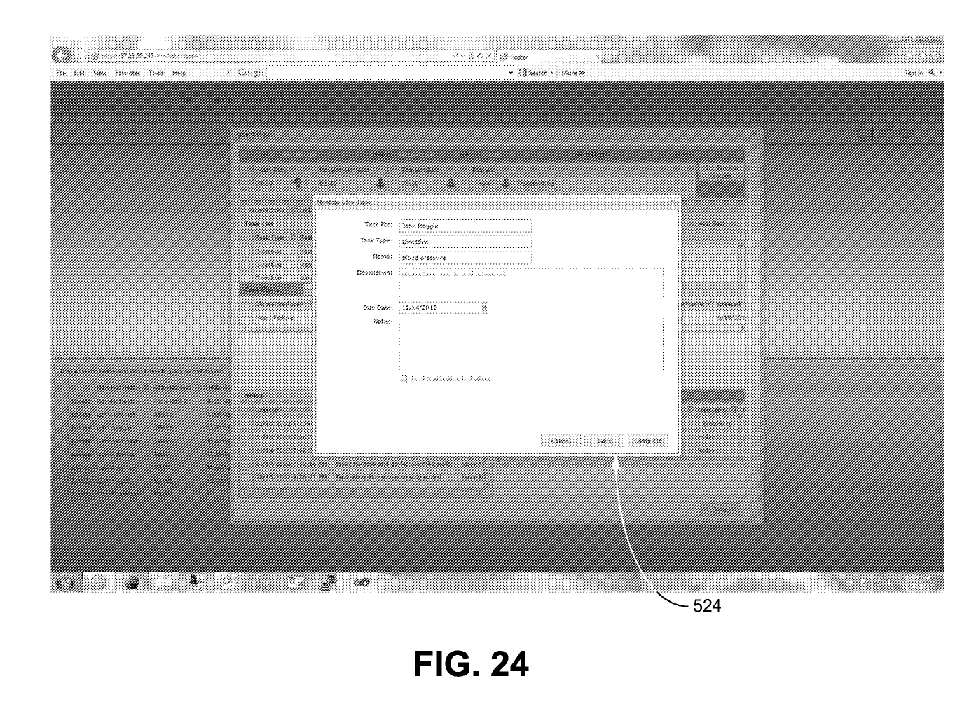

Tracking requests may be added, deactivated and/or modified by a medic, first responder, care giver or care provider through the patient tracker screen 518. For example, a selected tacking request may be modified through a patient tracker management screen 521 such as the example shown in FIG. 23. The patient tracker management screen 521 allows the medic, first responder, care giver or care provider to define tracking levels for vital signs of the associated user. Tasks (or actions) associated with the defined levels may also be set through this screen. The tasks (or actions) may be may be initiated in response to a comparison with a corresponding threshold value. By selecting the appropriate icon (e.g., an “add task” icon), a manage user task screen 524 such as the example of FIG. 24 may be accessed to allow the medic, first responder, care giver or care provider to define the task for the user (or patient) of the UE 103. The manage user task screen 524 allows the medic, first responder, care giver or care provider to enter, e.g., a user, task type, task name, task description, due date and/or time, and notes for the user.

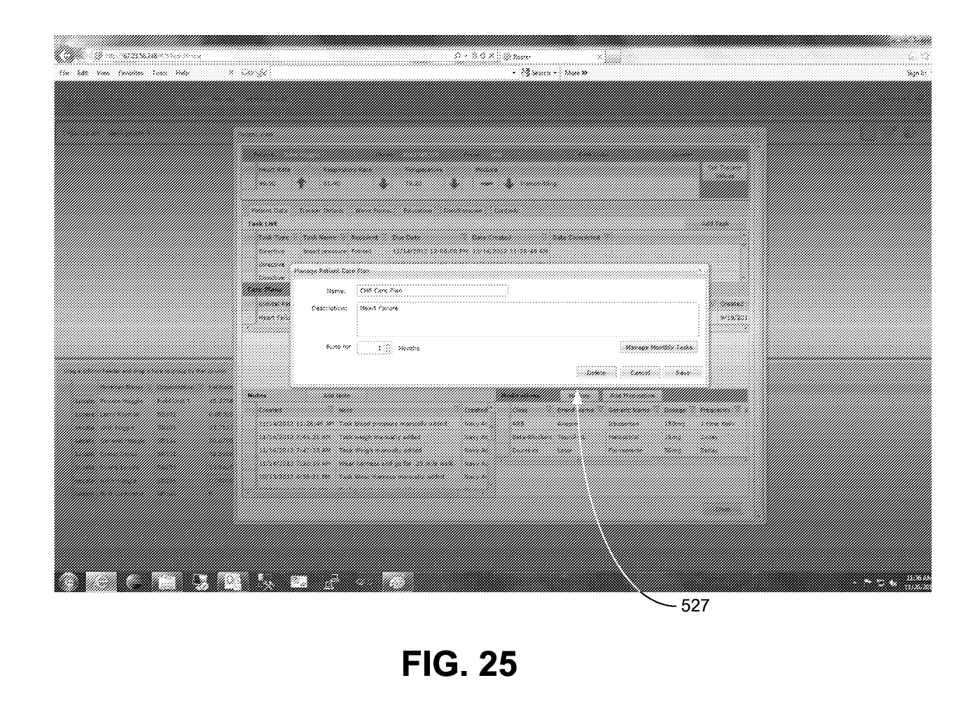

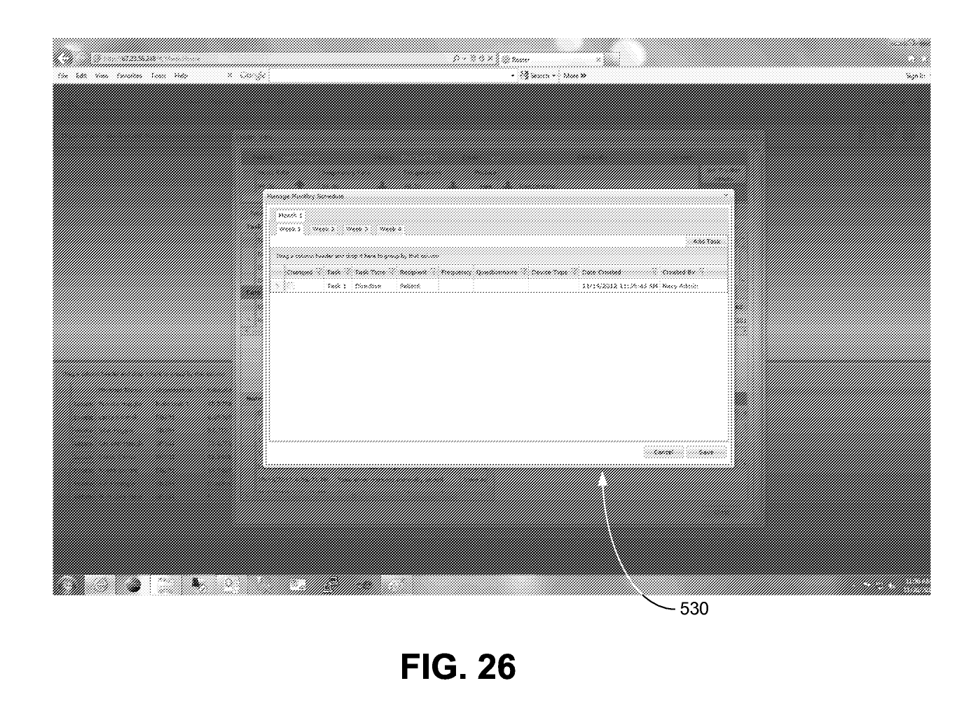

By selecting the appropriate icon in the patient view screen 515 of FIG. 21 (e.g., an “add care plan” icon), the medic, first responder, care giver or care provider can access a manage care plan screen to add and/or define a care plan for the user (or patient) being monitored. FIG. 25 shows an example of a manage care plan screen 527. The manage care plan screen 527 allows the medic, first responder, care giver or care provider to enter, e.g., a care plan name, care plan description, and care plan period (e.g., monthly, bimonthly, etc.) for the planned tasks. Existing care plans may be reviewed and/or modified by selecting the care plan listed in the patient view screen 515. The planned tasks may be managed by selecting an appropriate icon (e.g., a “manage monthly tasks” icon). The planned tasks may be reviewed, added and/or modified through a manage care plan schedule screen 530 such as the example of FIG. 26. The manage care plan schedule screen 530 allows the medic, first responder, care giver or care provider to enter and/or change tasks for the care plan. For example, a task, task type, recipient, frequency, questionnaire may be specified. In addition, the source and creating date and/or time of the task may be indicated.

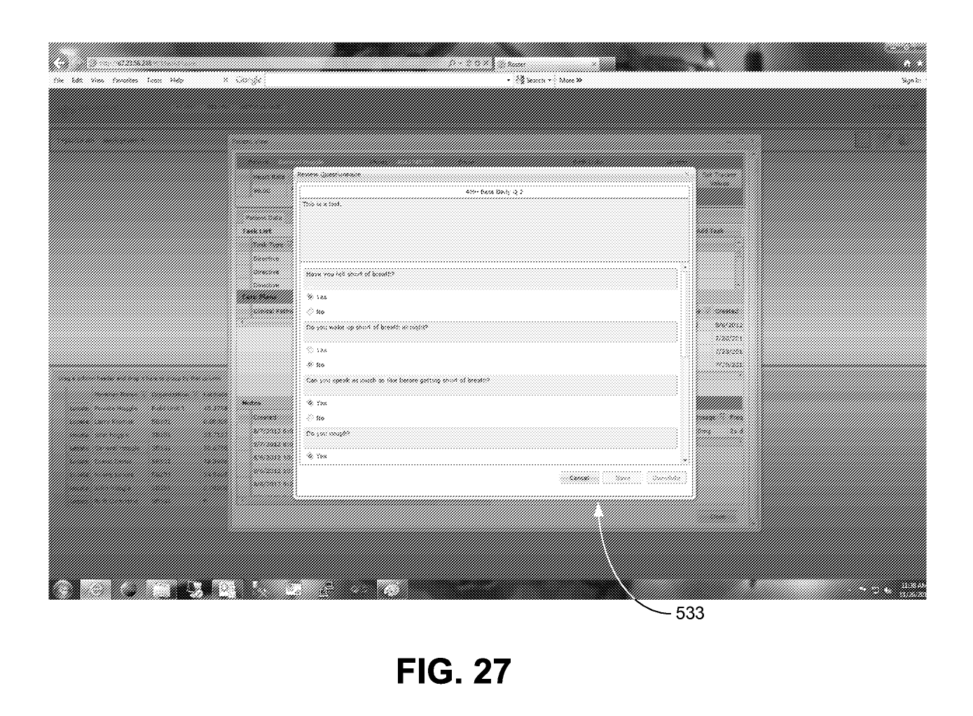

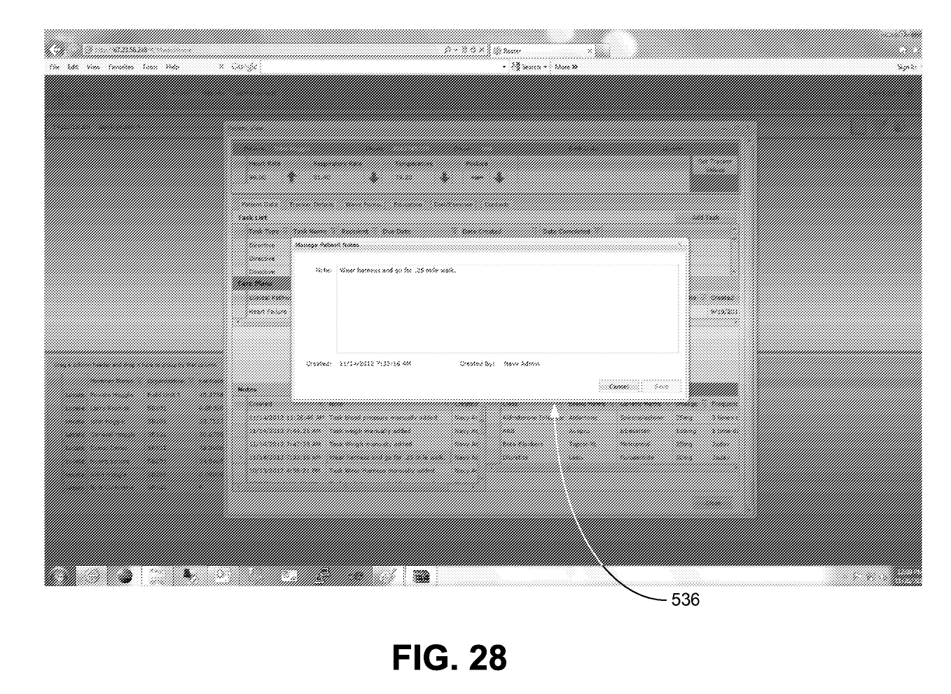

Questionnaires and/or notes may also be reviewed, added and/or modified through the patient view screen 515 of FIG. 21. By selecting the appropriate icon in the patient view screen 515 (e.g., an “add questionnaire” or “add note” icon), the medic, first responder, care giver or care provider can access a screen to add and/or define the item. Existing questionnaires and/or notes may be reviewed and/or modified by selecting the item listed in the patient view screen 515. FIG. 27 shows an example of a questionnaire screen 533, which allows the medic, first responder, care giver or care provider to define questions for the associated user and the type of response desired (e.g., yes/no, multiple choice, or short answers). The answers may then be used to determine condition of user. FIG. 28 shows an example of a manage notes screen 536, which allows the medic, first responder, care giver or care provider to write and save a note for the associated user. The notes can be included in the user's medical history.

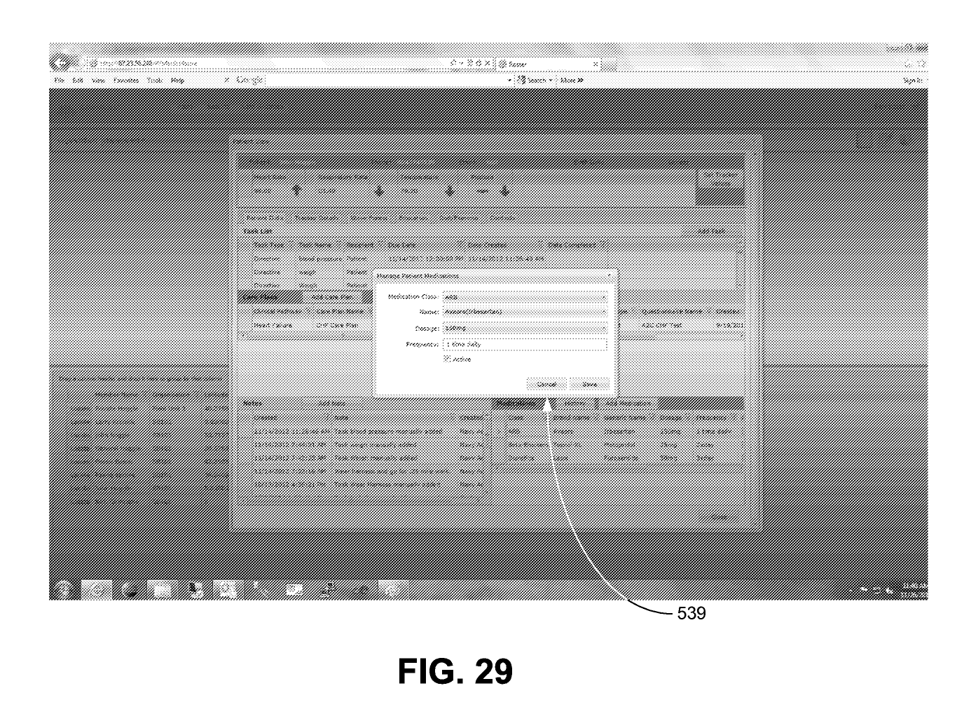

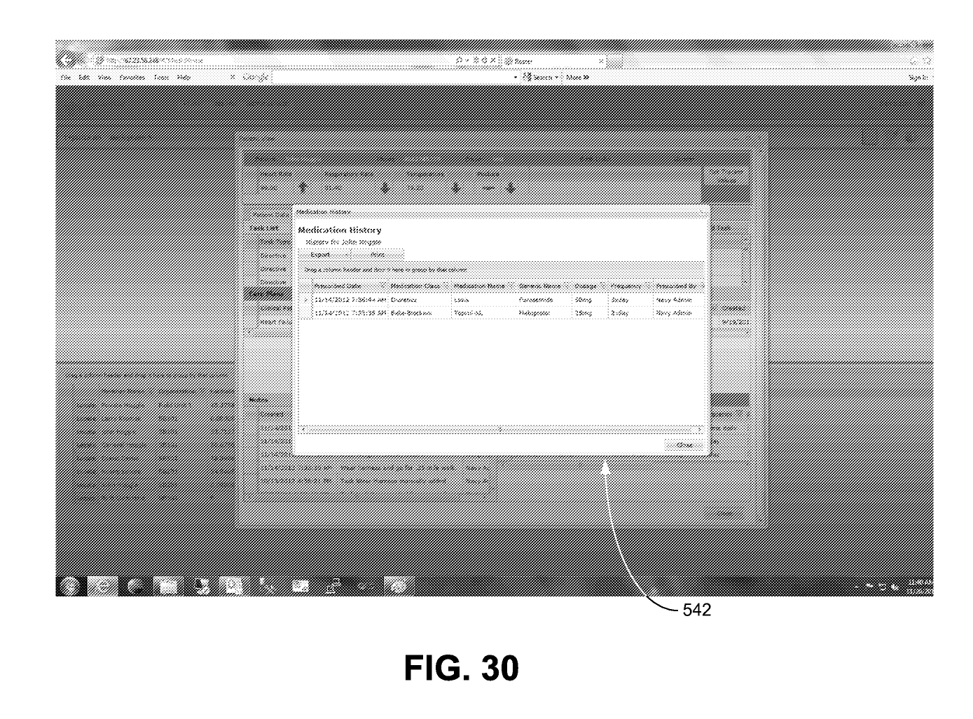

Medications of the monitored user may also be reviewed, added and/or modified through the patient view screen 515 of FIG. 21. By selecting the appropriate icon in the patient view screen 515 (e.g., an “add medication” or “history” icon), the medic, first responder, care giver or care provider can access a screen to review, add and/or modify a prescribed medication. Existing medications may be reviewed and/or modified by selecting the item listed in the patient view screen 515. For example, a medication may be added or modified through a manage medication screen 539 such as the example in FIG. 29. The manage medication screen 539 may allow for definition and/or modification of, e.g., the medication class and name, dosage and frequency, and/or whether the prescription is active. Medications may also be canceled through the manage medication screen 539. Access to the manage medication screen 539 may be limited to appropriate clinicians or doctors qualified to proscribe medications to patients. In some cases, additional verifications may be needed to access the manage medication screen 539. FIG. 30 shows an example of a medication history screen 542, which allows the medic, first responder, care giver or care provider to review medications that are or have been taken by the monitored user. The medication history screen 542 can include, e.g., prescription date, medication class and name, generic name if applicable, dosage and frequency, and the source that prescribed the medication.

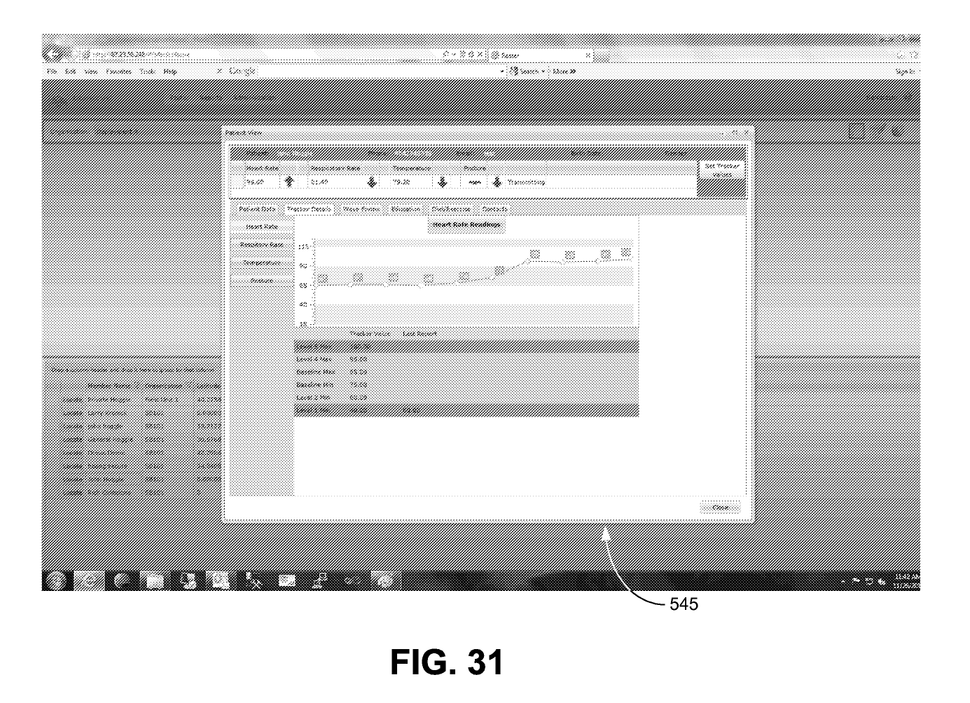

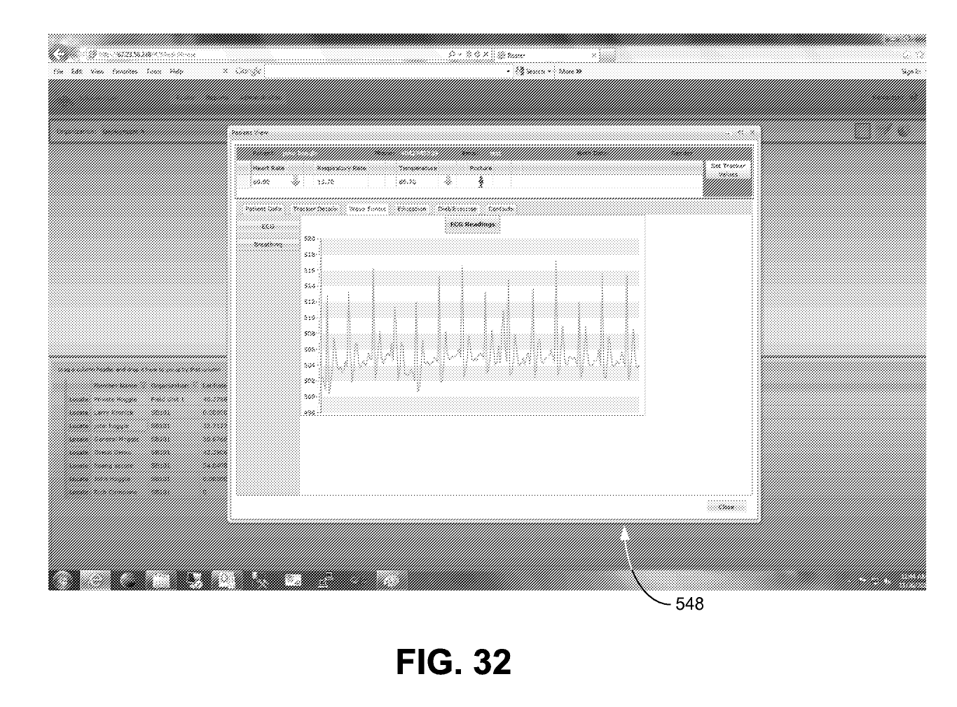

Other data such as, e.g., tracker details, captured waveforms, education, diet and/or exercise and/or contacts may also be accessed through, e.g., tabs in the patient view screen 515 of FIG. 21. For instance, FIG. 31 shows an example of a tracker details screen 545 that provides a graphical representation of a tracked vital sign such as, e.g., heart rate to allow the medic, first responder, care giver or care provider to compare measurements over time. The tracker details screen 545 can include limits (e.g., minimum and/or maximum levels or thresholds) for the tracked vital sign, which can be represented as highlighted regions in the graphical representation. Other tracked vital signs (e.g., respiratory rate, temperature, or posture) may be viewed by selecting the appropriate icon. FIG. 32 shows an example of a waveform screen 548 that allows the medic, first responder, care giver or care provider to view a captured vital sign waveform such as, e.g., an ECG or breathing pattern. The waveform may be viewed in real-time using data sent by the UE 103 of the user.

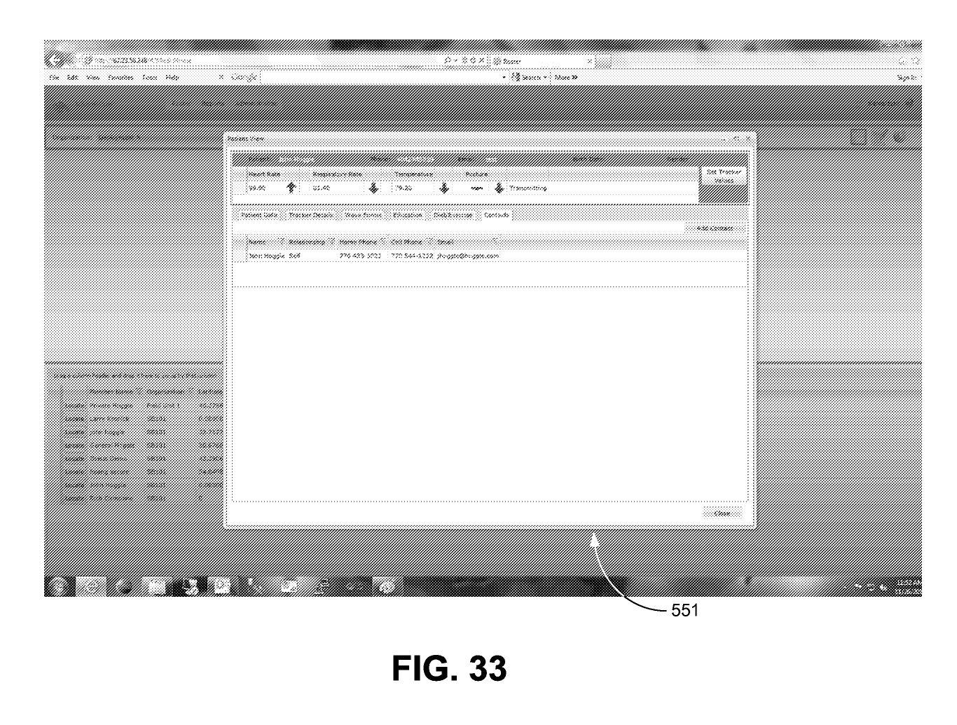

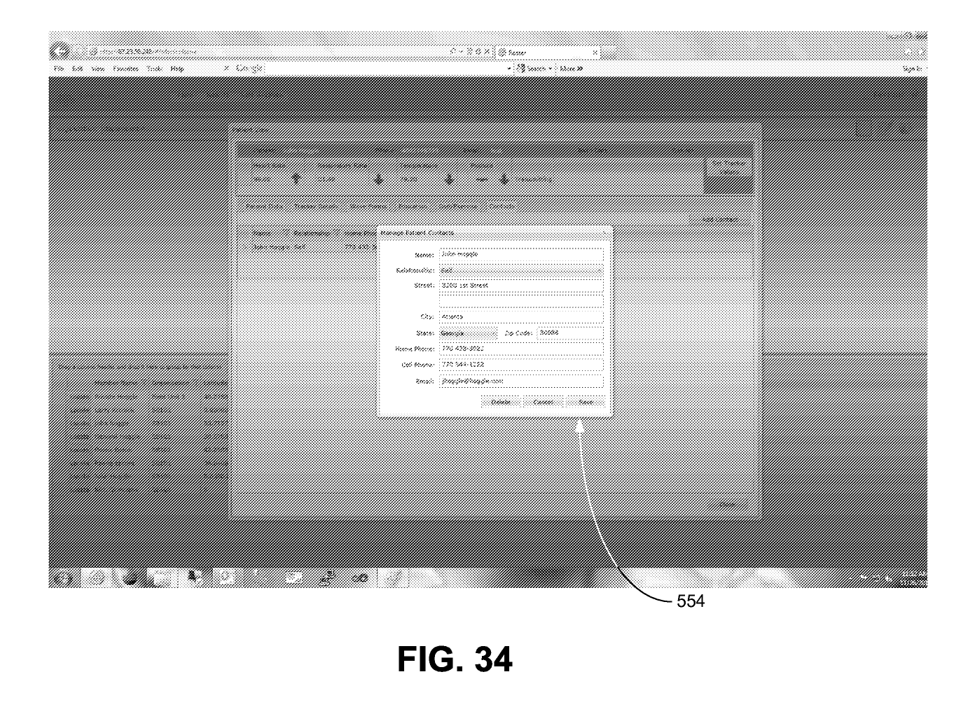

FIG. 33 shows an example of a contact screen 551 that allows the medic, first responder, care giver or care provider to view contact information of the associated user. The contact information can include, .e.g., contact name, relationship with the user, addresses, telephone numbers, or e-mail addresses associated with the user. Existing contacts may be reviewed and/or modified by selecting the contact listed in the contact screen 551. Contact information may be reviewed, modified and/or added through a manage contacts screen 554 such as the example shown in FIG. 34.

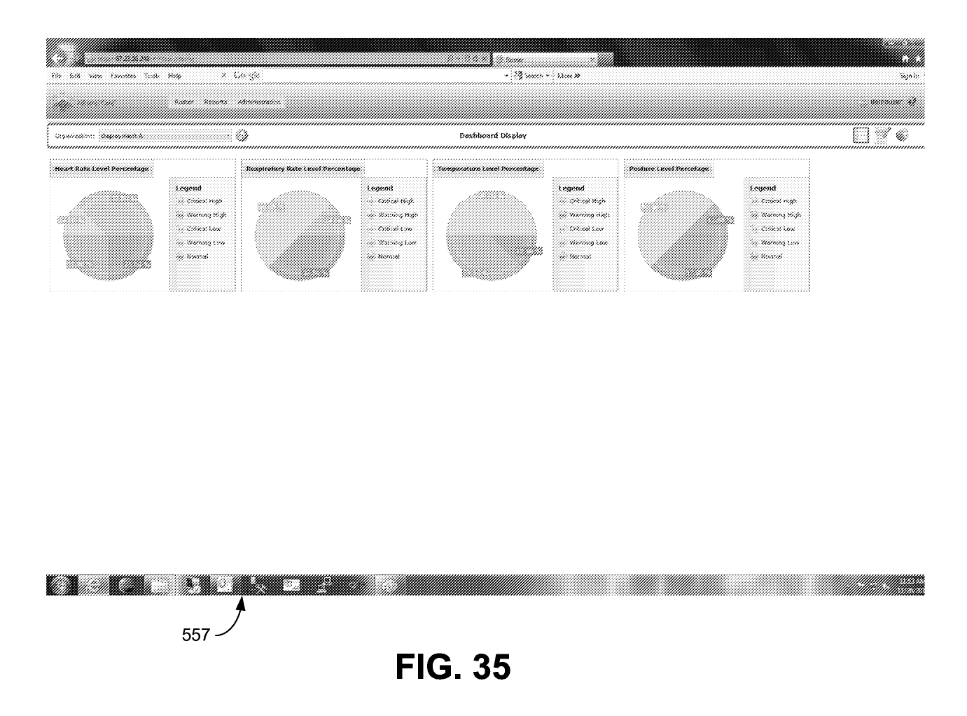

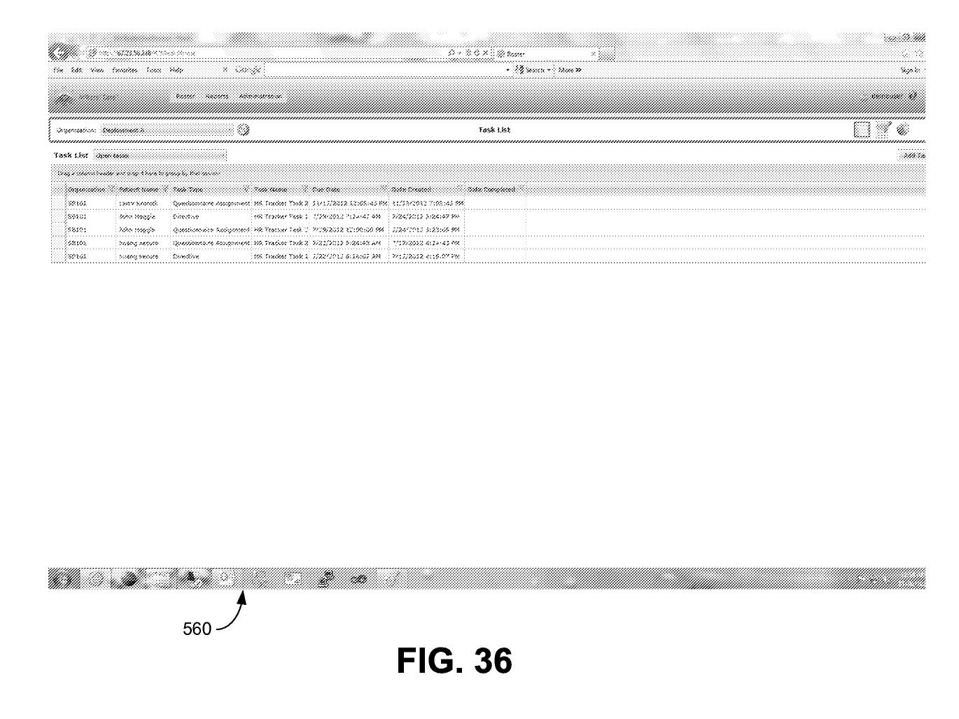

Other screens may also be presented to the medic, first responder, care giver or care provider through the web browser of the client device. For example, a dashboard screen 557 such as the example in FIG. 35 can provide graphical representations of the monitored vital signs of the monitored users. The dashboard screen 557 allows the medic, first responder, care giver or care provider to view a summary of status information of the users on the roster. The dashboard screen 557 may give an overview of the whole user population. Color coding provides a breakdown of the distribution of the vital signs of users (e.g., ranges of critical high, warning high, normal, warning low and critical low), which provides trends of the population. In addition, a roster task screen 560 such as the example in FIG. 36 may be the medic, first responder, care giver or care provider may allow the medic, first responder, care giver or care provider to view a summary of outstanding tasks for the users on the roster.

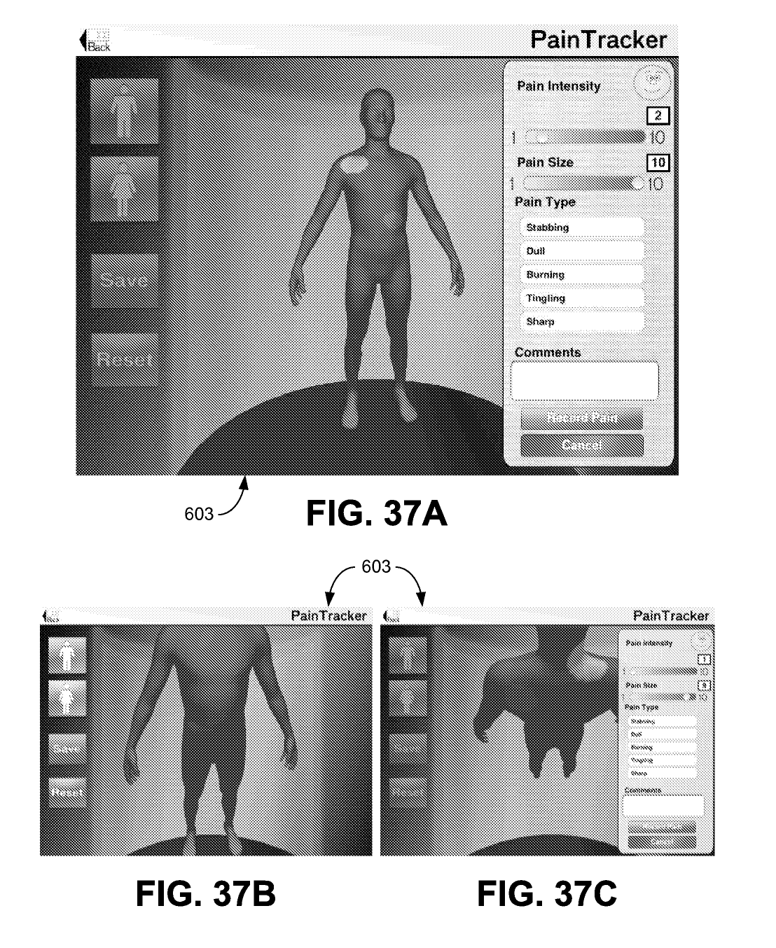

The pain of a user being monitored may also be indicated and/or tracked using the medical monitoring system 200 (FIG. 2). A pain tracking feature may be used as part of user interface of the client application on the UE 103 (FIGS. 1-3) to create a decision support tool. An example of a pain tracking screen 603 displayed on the UE 103 is shown in FIGS. 37A-37C. As shown in FIGS. 37A-37C, the pain tracking feature may use a three dimensional (3D) rotational model to track pain that the patient quantifies. Locations of the pain may be indicted by the user by selecting the appropriate area on the 3D rotational model. The 3D rotational model may be moved by the user to access the location of the pain as shown in FIGS. 37A-37C. Pain intensity, size and type may be specified through the pain tracking screen 603. Comments may also be entered by the user of the UE 103. In some cases, the pain tracking feature may be implemented as a separate client application running on the UE 103.

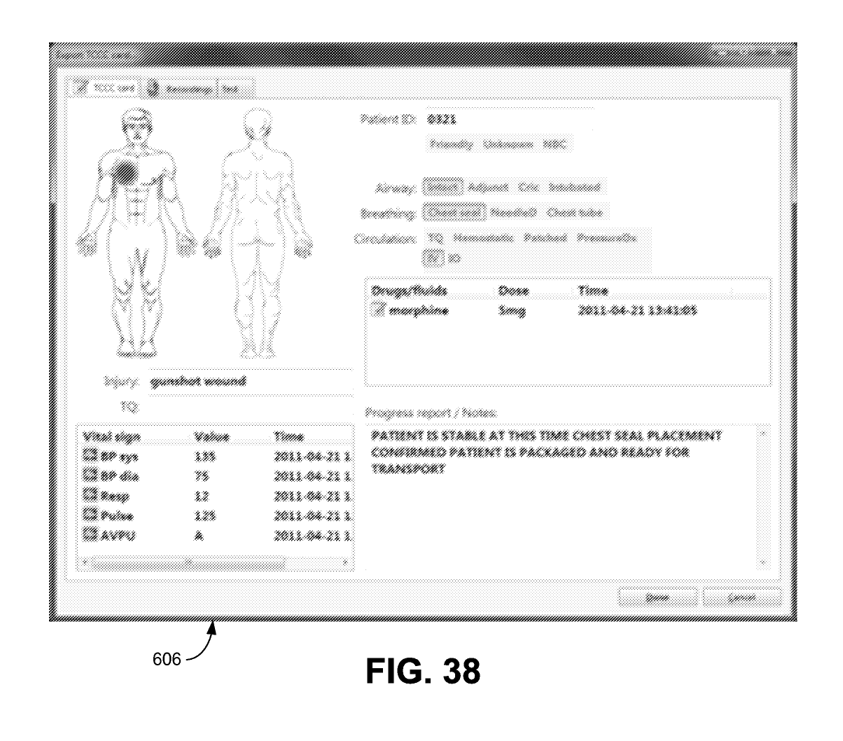

The medic, first responder, care giver or care provider may access the pain information through, e.g., a pain care screen 606 such as the example in FIG. 38, which mimics the paper tactical casualty care card (TC3) that is currently in use by the military. The pain care screen 606 can include indications of, e.g., the pain information from the pain tracking screen 603 (e.g., location, intensity, and size), type of injury, conditions of bodily systems (e.g., airway, breathing and circulation, and values of the monitored vital signs. The pain care screen 606 may also indicate any medications administered to the injured user by, e.g., the medic or first responder and any notes related to the condition or progress of the user. User identification can also be included. The pain care screen 606 can provide some knowledge of injury and/or treatment to the medic, first responder, care giver, care provider or other medical professionals upon the patient's arrival at a medical facility. The pain care screen 606 can electronically recreate the TC3 by modifying some of the information obtained through the pain tracking screen 603 (FIGS. 37A-37C). The pain tracking screen 603 can be used in the medical monitoring system 200 to automatically capture the patient's vital signs to populate pain care screen 606.

Predictive models may be used to correlate data from the user's vital signs and information obtained through the pain tracking feature to indicate a course of action for the medic or first responder before putting an injured user (or wounded soldier) onto a manned or unmanned medical transport vehicle. The output provided to the medic or first responder may be a medical course of action derived from, e.g., the Army Medical Handbook. In addition, a go/no-go indicator may be provided to tell the medic or first responder whether or not they need to ride in the medical transport vehicle with the patient. For example, the go/no-go indicator may indicate that a medic should travel with the patient in an unmanned vehicle or that the patient should be all right until the unmanned vehicle reaches its destination with the injured user.

Medical telemetry can result in appropriate intervention and treatment. The medical monitoring system 200 allows for remote physiological monitoring, data basing, and medical information exchange among patient, medics, first responders, care givers and/or remote care providers. Semi-autonomous, autonomous, or closed-loop monitoring, treatment and intervention systems, addressing lifesaving monitoring and treatment in route from the point of injury through the evacuation process to the medical facility via manned or unmanned medevac vehicles or even standard military or civilian vehicles transport may be possible.

The medical monitoring system 200 can support in route automated combat casualty care from the point of injury to a local or remote medical facility.

The platform can operate either on a 3G/4G cellular network, other broadband network or a tactical radio network. The medical monitoring system 200 provides the capability for physiological vital sign monitoring with telemetry, medical information exchange and integration with semi-autonomous, autonomous or closed loop treatment systems at the point of injury and while in transport either on manned or unmanned vehicles. Assessment tools, questionnaires, pain tracking features, and predictive models for rapid triage and diagnosis plus diagnostic imaging can be supported by the medical monitoring system 200. Treatment guidelines and interventions for injuries such as loss or near loss of limb, blocked air way, sucking chest wound, internal bleeding or traumatic brain injury can be communicated to a medic or first responder through the medical monitoring system 200.

Applications can be deployed on ruggedized local servers housed at or near the communication network hub. With the availability of secure back haul networks, virtual servers may be employed. Ruggedized user equipment (UE) 103 may be utilized to improve reliability of the medical monitoring system 200. For example, smartphones can have a hard rubber coating which will provide protection from breakage, dust and other elements of a harsh environment. The smartphones can be loaded with the client application and may be enhanced for easy viewing in extreme light conditions. The smartphones may be compatible with any number of available headsets for hand free operation. Similarly, ruggedized tablets may be available for use and can provide may of the same features as a smartphone but on a larger easier to read screen.

The medical monitoring system 200 provides a cost effective way to provide fast and efficient remote collection, wireless transfer, aggregation and reporting of individually identifiable vital readings. The medical monitoring system 200 is suited for hospitals and health professionals, including cardiac care providers, military combat and training monitoring and medical response, police, fire and rescue monitoring and tracking, and in-home monitoring of chronically ill patients and anyone who may need the security and comfort of assistance with medication, diet, exercise and general oversight. Users of mobile vital signs monitoring devices can range from: (a) people suffering from a chronic health conditions; to (b) healthy individuals focused on improving their health or sports performance; to (c) remotely located family members concerned about monitoring an elderly parent.

For example, the medical monitoring system 200 can assist health care providers in reducing unnecessary hospital readmissions and meeting new quality of care guidelines. Following a patient's hospital stay or in response to a specific medical event, health care professionals may utilize the medical monitoring system 200 to provide remote monitoring services for a patient such as, e.g., a cardiac care patient. Health care professionals can utilize the medical monitoring system 200 both to reduce response time in the event of post-discharge medical emergencies and also to provide cost effective monitoring for the avoidance of unnecessary and expensive re-admissions. The medical monitoring system 200, along with hospitals and health care facilities, may provide real-time, web-based aggregation and reporting of vital medical data to qualified health care professionals who are then able to manage multiple patients with maximum efficiency.

In military combat and training environments, the medical monitoring system 200 operating on a portable network, can provide real-time assessment of vital medical information and individual GPS location capabilities. The medical monitoring system 200 can improve field communications; facilitate training exercise monitoring; and simulate combat environment triage prioritization in order to improve the speed and efficiency of search and rescue operations. In this way, the medical monitoring system 200 can increase the efficiency of identifying, rescuing, routing and treating the wounded.

The medical monitoring system 200 may be used by police, fire, homeland security and other rescue operations to coordinate and prioritize medical response in the event of an emergency, but may also provide real-time physical tracking and location of the first responders in the field. For example, the medical monitoring system 200 may be used to monitor fire fighters engaged in fighting wild fires in the wilderness. The GPS locational data can aid in first responder management and coordination, while the Bluetooth® communication capability significantly reduces the time necessary to diagnose and treat the injured user, by providing real-time medical information and communications between field locations and hospital based health care professionals.

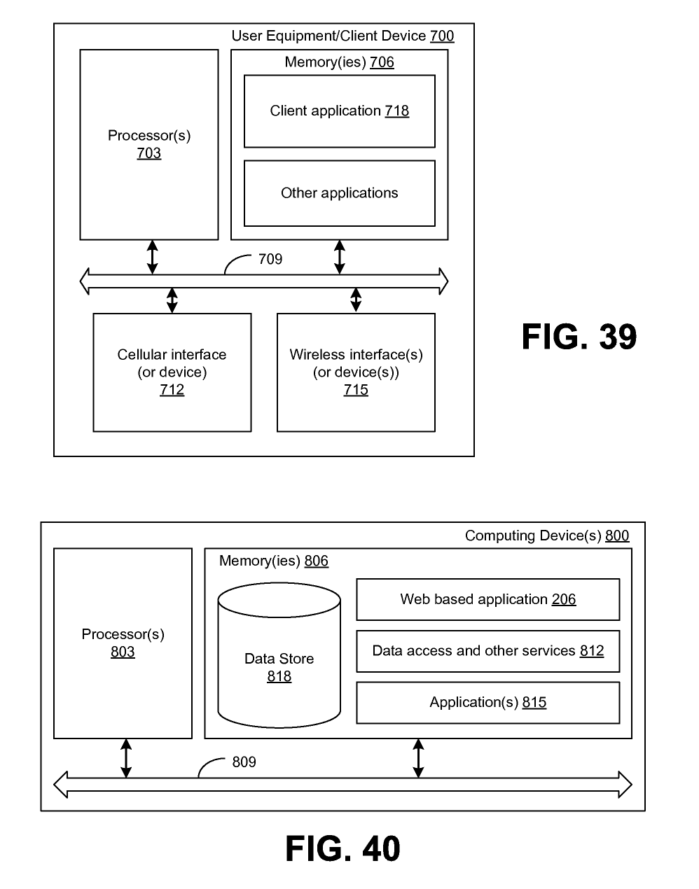

With reference to FIG. 39, shown is a schematic block diagram of an example of user equipment (UE) or client device 700 in accordance with various embodiments of the present disclosure. The user equipment (UE) or client device 700includes at least one processor circuit, for example, having a processor 703 and a memory 706, both of which are coupled to a local interface 709. The user equipment (UE) or client device 700 may include a cellular interface 712 such as, e.g., an LTE interface and one or more wireless interface 715 including, e.g., a BT interface, all of which may be coupled to the local interface 709. The cellular interface 712 comprises processing circuitry for supporting cellular communications such as, e.g., LTE, 2G, 3G, 4G, WiMAX, WCDMA, HSDPA, or other wireless communication protocols. The wireless interface(s) 715 comprise processing circuitry for supporting wireless communications such as, e.g., Bluetooth (BT), IEEE 802.11 a/b/g/n, near field communication (NFC), global positioning system (GPS)/global navigation satellite system (GNSS), and/or other wireless communication protocols.

In various embodiments, the processing circuitry is implemented as at least a portion of a microprocessor. The processing circuitry may be implemented using one or more circuits, one or more microprocessors, application specific integrated circuits, dedicated hardware, digital signal processors, microcomputers, central processing units, field programmable gate arrays, programmable logic devices, state machines, or any combination thereof. In yet other embodiments, the processing circuitry may include one or more software modules executable within one or more processing circuits. The processing circuitry may further include memory configured to store instructions and/or code that causes the processing circuitry to execute data communication functions. In some cases, portions of the cellular interface 712 and/or wireless interface(s) 715 may be implemented by processor 703 via local interface 709. The local interface 709 may comprise, for example, a data bus with an accompanying address/control bus or other bus structure as can be appreciated.

Stored in the memory 706 are both data and several components that are executable by the processor 703 and/or by processing circuitry of the cellular interface 712 and/or wireless interface(s) 715. In particular, stored in the memory 706 and executable by the processor 703 may be a client application 718 for the medical monitoring system 200, and potentially other applications. In addition, an operating system may be stored in the memory 706 and executable by the processor 803. It is understood that there may be other applications that are stored in the memory and are executable by the processor 703, the cellular interface 712 and/or wireless interface(s) 715 as can be appreciated.

With reference now to FIG. 40, shown is a schematic block diagram of a computing device 800 according to an embodiment of the present disclosure. The computing device 800 includes at least one processor circuit, for example, having a processor 803 and a memory 806, both of which are coupled to a local interface 809. To this end, the computing device 800 may comprise, for example, at least one server computer or like device. The local interface 809 may comprise, for example, a data bus with an accompanying address/control bus or other bus structure as can be appreciated.

Stored in the memory 806 are both data and several components that are executable by the processor 803. In particular, stored in the memory 806 and executable by the processor 803 are the web based application 206 (FIG. 2), data access and other services 812 available through the intermediate services layer 212 (FIG. 2), and potentially other applications 815. Also stored in the memory 806 may be a data store 818 including, e.g., a secure database 215 (FIG. 2) and other data. In addition, an operating system may be stored in the memory 806 and executable by the processor 803. It is understood that there may be other applications that are stored in the memory and are executable by the processor 803 as can be appreciated.

Referring to both FIGS. 39 and 40, where any component discussed herein is implemented in the form of software, any one of a number of programming languages may be employed such as, for example, C, C++, C#, Objective C, Java®, JavaScript , Perl, PHP, Visual Basic®, Python®, Ruby, Delphi®, Flash®, or other programming languages. A number of software components are stored in the memory and are executable by the processor 703/803, the cellular interface 712 and/or wireless interface(s) 715. In this respect, the term “executable” means a program file that is in a form that can ultimately be run by the processor 703/803, the cellular interface 712 and/or wireless interface(s) 715. Examples of executable programs may be, for example, a compiled program that can be translated into machine code in a format that can be loaded into a random access portion of the memory 706/806 and run by the processor 703/803, source code that may be expressed in proper format such as object code that is capable of being loaded into a random access portion of the memory 706/806 and executed by the processor 703/803, or source code that may be interpreted by another executable program to generate instructions in a random access portion of the memory 706/806 to be executed by the processor 703/803, etc. An executable program may be stored in any portion or component of the memory including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory is defined herein as including both volatile and nonvolatile memory and data storage components. Volatile components are those that do not retain data values upon loss of power. Nonvolatile components are those that retain data upon a loss of power. Thus, the memory 706/806 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 703/803 may represent multiple processors 703/803 and the memory 706/806 may represent multiple memories 706/806 that operate in parallel processing circuits, respectively. In such a case, the local interface 709/809 may be an appropriate network that facilitates communication between any two of the multiple processors 703/803, between any processor 703/803 and any of the memories 706/806, or between any two of the memories 706/806, etc. The local interface 709/809 may comprise additional systems designed to coordinate this communication, including, for example, performing load balancing. The processor 703/803 may be of electrical or of some other available construction.

Although portions of the medical monitoring system 200, and other various systems described herein may be embodied in software or code executed by general purpose hardware, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits having appropriate logic gates, or other components, etc. Such technologies are generally well known by those skilled in the art and, consequently, are not described in detail herein.

The medical monitoring system 200 can comprise program instructions to implement logical function(s) and/or operations of the system. The program instructions may be embodied in the form of source code that comprises human-readable statements written in a programming language or machine code that comprises numerical instructions recognizable by a suitable execution system such as a processor 703/803 in a computer system or other system. The machine code may be converted from the source code, etc. If embodied in hardware, each block may represent a circuit or a number of interconnected circuits to implement the specified logical function(s).

Also, any logic or application described herein, including the medical monitoring system 200 that comprises software or code can be embodied in any non-transitory computer-readable medium for use by or in connection with an instruction execution system such as, for example, a processor 703/803 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

Therefore, at least the following is claimed:

1. A system for medical monitoring, comprising:
a monitoring device capable of obtaining one or more vital signs of a user;
user equipment communicatively coupled to the monitoring device, the user equipment capable of obtaining the one or more vital signs from the monitoring device and providing the one or more vital signs to a remote server for access by a care giver.

2. The system of claim 1, wherein the user equipment is a smartphone.

3. The system of claim 1, wherein the user equipment is communicatively coupled to the monitoring device via a Bluetooth connection.

4. The system of claim 1, wherein the user equipment securely transmits the one or more vital signs to the remote server via a link through a cellular network.

5. The system of claim 4, wherein the link is established through a mobile node of the cellular network.

6. The system of claim 4, wherein the user equipment is capable of transmitting real-time indications of the one or more vital signs obtained from the monitoring device to the care giver.

7. The system of claim 6, wherein the care giver is a medic or a first responder.

8. The system of claim 6, wherein the user equipment is capable of providing a current location of the user to the care giver.

9. The system of claim 8, wherein the current location of the user is provided to a medical transport in route to the user.

10. The system of claim 1, wherein the one or more vital signs are stored in a database of the remote server for subsequent access by the care giver.

11. The system of claim 1, wherein the user equipment is capable of presenting a task for the user from the care giver, the task based at least in part upon the one or more vital signs provided by the user equipment.

12. The system of claim 11, wherein the task is further based at least in part based upon responses of the user to questions from the care giver regarding a medical condition of the user, the questions presented to the user through a questionnaire displayed on the user equipment.

13. The system of claim 11, wherein the task comprises taking a medication prescribed by the care giver.

14. The system of claim 13, wherein the care giver is a clinician at a remote medical facility.

15. The system of claim 1, wherein the user equipment comprises a pain tracking interface configured to allow the user to indicate location and intensity of pain experienced by the user.
